(19) United States
(12) Patent Application Publication
(10) Pub. No.: US 2003/0133913 A1
(43) Pub. Date: Jul. 17, 2003

(54) Methods of maturing plasmacytoid dendritic cells using immune response modifier molecules

(75) Inventors: Mark A. Tomai, Woodbury, MN (US); John P. Vasilakos, Woodbury, MN (US); John C. Stolpa, St. Paul, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/229,829

(22) Filed: Aug. 28, 2002

Related U.S. Application Data
(60) Provisional application No. 60/316,144, filed on Aug. 30, 2001.
(60) Provisional application No. 60/370,177, filed on Apr. 5, 2002.

(51) Int. Cl. G01N 33/567; A61K 45/00; A61K 31/4745; C12N 5/08

(52) U.S. Cl. 424/93.7; 435/7.21; 435/372; 514/292; 514/291

(57) ABSTRACT

The present invention relates to methods of maturing plasmacytoid dendrites cells using immune response modifier molecules. The present invention also relates to methods of detecting biological activities of matured plasmacytoid dendritic cells and methods of using mature plasmacytoid dendritic cells for therapeutic or prophylactic purposes.

This application claims the benefit of U.S. Provisional Patent Application Ser. Nos. 60/316,144, filed Aug. 30, 2001 and 60/370,177, filed Apr. 5, 2002.

### BACKGROUND OF THE INVENTION

Dendritic cells are antigen-presenting cells of the immune system that provide a functional bridge between the innate and the acquired immune systems. Immature dendritic cells can reside in various tissues of the body, where they may encounter pathogens or other foreign antigens. These encounters induce the secretion of certain cytokines including, for example, interferons such as IFN-α. The immature dendritic cells may capture an antigen and then migrate to lymphoid tissue where, after the dendritic cells mature, they present the antigen (or a portion of the antigen) to lymphocytes. Antigen presentation triggers parallel immunological cascades resulting in an antigen-specific cell-mediated immune response and an antigen-specific humoral immune response.

Plasmacytoid dendritic cells (pDCs) have been identified as the primary class of dendritic cell responsible for producing and secreting interferons, including IFN-α, in response to an immunological challenge. A class of compounds known as immune response modifiers (IRMs) also can induce the production of various cytokines, including IFN-α; in numerous species, including humans.

Certain IRMs are small organic molecules such as those disclosed in, for example, U.S. Pat. Nos. 4,689,338; 4,929,624; 5,266,575; 5,268,376; 5,352,784; 5,389,640; 5,482,936; 5,494,916; 6,110,929; 6,194,425; 4,988,815; 5,175,296; 5,367,076; 5,395,937; 5,693,811; 5,741,908; 5,238,944; 5,939,090; 6,245,776; 6,039,969; 6,083,969; 6,245,776; 6,331,539; and 6,376,669; and PCT Publications WO 00/76505; WO 00/76518; WO 02/46188, WO 02/46189; WO 02/46190; WO 02/46191; WO 02/46192; WO 02/46193; and WO 02/46194. Additional small molecule IRMs include purine derivatives (such as those described in U.S. Pat. Nos. 6,376,501 and 6,028,076), small heterocyclic compounds (such as those described in U.S. Pat. No. 6,329,381), and amide derivatives (such as those described in U.S. Pat. No. 6,069,149). Some of these small molecule IRMs may act through one or more Toll-like receptors (TLR) such as, for example, TLR-1, TLR-2, TLR-4, TLR-6, TLR-7, and TLR-8.

Other IRMs include large biological molecules such as oligonucleotide sequences. Some IRMs oligonucleotide sequences contain cytosine-guanine dinucleotides (CpG) and are described, for example, in U.S. Pat. Nos. 6,194,388; 6,207,646; 6,239,116; 6,339,068; and 6,406,705. CpG has been reported to act through TLR 9. Further, CpG molecules may be used to activate dendritic cells (see, e.g., U.S. Pat. No. 6,429,199). Other IRM nucleotide sequences lack CpG and are described, for example, in International Patent Publication No. WO 00/75304.

### SUMMARY OF THE INVENTION

The present invention provides a method of inducing antigen presentation by dendritic cells in vitro, the method including: (a) exposing an isolated dendritic cell population to an antigen; (b) contacting the isolated dendritic cell with an immune response modifier molecule that is an agonist of Toll-like receptor 6, Toll-like receptor 7 or Toll-like receptor 8; and (c) allowing the dendritic cell to process and present the antigen. In this aspect of the invention and in all additional aspects that follow, for some embodiments the immune response modifier molecule is an agonist of Toll-like receptor 7, and in other embodiments, the immune response modifier molecule is selected from the group consisting of imidazoquinoline amines, imidazopyridine amines, 6,7-fused cycloalkylimidazopyridine amines, 1,2-bridged imidazoquinoline amines, thiazolo- and oxazolo-quinolinamines and pyridinamines, imidazonaphthyridine amines and tetrahydroimidazonaphthyridine amines, and pharmaceutically acceptable salts thereof.

In another aspect, the present invention provides a method of detecting cytokine production by a plasmacytoid dendritic cell, the method including: (a) contacting an isolated plasmacytoid dendritic cell with an immune response modifier molecule that is an agonist of Toll-like receptor 6, Toll-like receptor 7 or Toll-like receptor 8 in an amount effective for inducing the plasmacytoid dendritic cell to produce one or more cytokines selected from IL-8, IP-10, IL-6, MIP-1α, and IFN-ω; and (b) detecting production of at least one of the cytokines by the dendritic cell.

In another aspect, the present invention provides a method of detecting expression of co-stimulatory markers by plasmacytoid dendritic cells, the method including: (a) contacting an isolated plasmacytoid dendritic cell with an immune response modifier molecule that is an agonist of Toll-like receptor 6, Toll-like receptor 7 or Toll-like receptor 8 in an amount effective for inducing the plasmacytoid dendritic cell to express one or more co-stimulatory marker; and (b) detecting the expression of at least one co-stimulatory marker by the plasmacytoid dendritic cell.

In another aspect, the present invention provides a method of enhancing survival of isolated plasmacytoid dendritic cells, the method including: (a) contacting a population of isolated plasmacytoid dendritic cells with an immune response modifier molecule that is an agonist of Toll-like receptor 6, Toll-like receptor 7 or Toll-like receptor 8 in an amount effective for enhancing survival of the plasmacytoid dendritic cells; and (b) incubating the plasmacytoid dendritic cells under conditions so that at least 30% of the plasmacytoid dendritic cell survive for at least 48 hours.

In another aspect, the present invention provides a method of detecting expression of chemokine receptors by plasmacytoid dendritic cells, the method including: (a) contacting an isolated plasmacytoid dendritic cell with an immune response modifier molecule that is an agonist of Toll-like receptor 6, Toll-like receptor 7 or Toll-like receptor 8 in an amount effective for inducing the plasmacytoid dendritic cell to express one or more chemokine receptor; and (b) detecting expression of at least one chemokine receptor.

In another aspect, the present invention provides a method of identifying a compound that selectively induces production of a chemokine receptor by plasmacytoid dendritic cells, the method including: (a) obtaining a population of cells that includes both inflammatory cytokine producing cells and plasmacytoid dendritic cells; (b) contacting the population of cells with a test compound; (c) determining the amount of chemokine receptor present in the population of cells contacted with the test compound; (d) determining the amount of inflammatory cytokine(s) present in the population of cells contacted with the test compound; and (e) identifying the test compound as a selective inducer of the chemokine receptor if the chemokine receptor is present in the population of cells after contact with the test compound in an amount at least three times greater than the amount of inflammatory cytokine(s) present in the population of cells.

In another aspect, the present invention provides a method of preparing a cell population enriched for cells that express a chemokine receptor, the method including: (a) contacting an isolated plasmacytoid dendritic cell with an immune response modifier molecule that is an agonist of Toll-like receptor 6, Toll-like receptor 7 or Toll-like receptor 8 in an amount effective for inducing the plasmacytoid dendritic cell to express one or more chemokine receptor; and (b) enriching the cell population for cells that express a chemokine receptor.

In another aspect, the present invention provides a method of treating a disease including: (a) contacting an isolated plasmacytoid dendritic cell with an immune response modifier molecule that is an agonist of Toll-like receptor 6, Toll-like receptor 7 or Toll-like receptor 8 in an amount effective for inducing the plasmacytoid dendritic cell to express one or more chemokine receptor; (b) contacting the population of plasmacytoid dendritic cells with an antigen associated with the disease; (c) enriching the cell population for cells expressing a high level of expression of at least one chemokine receptor; and (d) administering the enriched cell population to a patient.

In another aspect, the present invention provides a method of preparing a cellular adjuvant for the treatment of a disease including: (a) maturing plasmacytoid dendritic cells in vitro by treating the dendritic cells with an immune response modifying compound that is an agonist of Toll-like receptor 6, Toll-like receptor 7 or Toll-like receptor 8; and (b) exposing the mature dendritic cells to an antigen associated with said disease.

In another aspect, the present invention provides a method of treating a disease including administering a therapeutically effective dose of plasmacytoid dendritic cells that have been matured by stimulation with an immune response modifying compound that is an agonist of Toll-like receptor 6, Toll-like receptor 7 or Toll-like receptor 8 to mammal in need of such treatment.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1 shows ELISA detection of IFN-γ produced by T-cells as a result of antigen presentation by pDCs.

FIG. 2 shows ELISA detection of IL-10 produced by T-cells as a result of antigen presentation by pDCs.

FIG. 3 shows flow cytometry data comparing co-stimulatory marker expression by pDCs treated with IL-3, IFN-α and IRM.

FIG. 4 shows flow cytometry data comparing survival of pDCs when incubated with and without IRM.

FIG. 5 shows flow cytometry data comparing chemokine receptor CCR7 expression by pDCs treated with IL-3, IFN-α and IRM.

### DETAILED DESCRIPTION OF ILLUSTRATIVE EMBODIMENTS OF THE INVENTION

We have found that IRMs that are agonists of certain Toll-like receptors (for example, TLR-6 and TLR-7) can induce a variety of biological responses from pDCs in addition to the previously known response of producing IFN-α, For example, certain IRMs that are known to be agonists of TLR-6, TLR-7 or TLR-8 can induce human pDCs to produce cytokines such as IFN-ω and human inducible protein (IP)-10. These same IRMs also can enhance pDC (1) viability, (2) expression of co-stimulatory markers, (3) expression of chemokine receptors, and (4) antigen presentation, as measured by production of IFN-γ and IL-10 by naïve CD4⁺ T-cells, induced by contact with antigen presenting pDCs.

Plasmacytoid dendritic cells that exhibit increased expression of markers such as co-stimulatory markers or chemokine receptors may be enriched in a cell population. The enriched cell population may be used to produce one or more desired molecules in vitro that may subsequently be administered to a patient for therapeutic or prophylactic purposes. Alternatively, the enriched cell population itself may be administered to a patient for therapeutic or prophylactic purposes.

IRM Compounds

As noted above, many imidazoquinoline amine, imidazopyridine amine, 6,7-fused cycloalkylimidazopyridine amine, 1,2-bridged imidazoquinoline amine, thiazolo- and oxazolo-quinolinamines and pyridinamines, imidazonaphthyridine and tetrahydroimidazonaphthyridine amine IRM compounds have demonstrated significant immunomodulating activity. Exemplary immune response modifier compounds suitable for use in invention include 1H-imidazo[4,5-c]quinolin-4-amines defined by one of Formulas I-V below:

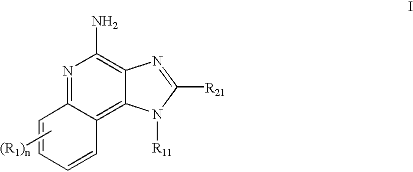

wherein

R₁₁ is selected from the group consisting of alkyl of one to ten carbon atoms, hydroxyalkyl of one to six carbon atoms, acyloxyalkyl wherein the acyloxy moiety is alkanoyloxy of two to four carbon atoms or benzoyloxy, and the alkyl moiety contains one to six carbon atoms, benzyl, (phenyl)ethyl and phenyl, said benzyl, (phenyl)ethyl or phenyl substituent being optionally substituted on the benzene ring by one or two moieties independently selected from the group consisting of alkyl of one to four carbon atoms, alkoxy of one to four carbon atoms and halogen, with the proviso that if said benzene ring is substituted by two of said moieties, then said moieties together contain no more than six carbon atoms;

R₂₁ is selected from the group consisting of hydrogen, alkyl of one to eight carbon atoms, benzyl, (phenyl)ethyl and phenyl, the benzyl, (phenyl)ethyl or phenyl substituent being optionally substituted on the benzene ring by one or two moieties independently selected from the group consisting of alkyl of one to four carbon atoms, alkoxy of one to four carbon atoms and halogen, with the proviso that when the benzene ring is substituted by two of said moieties, then the moieties together contain no more than six carbon atoms; and

each R₁ is independently selected from the group consisting of alkoxy of one to four carbon atoms, halogen, and alkyl of one to four carbon atoms, and n is an integer from 0 to 2, with the proviso that if n is 2, then said R₁ groups together contain no more than six carbon atoms;

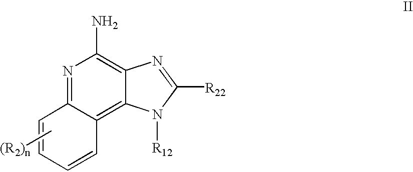

wherein

R₁₂ is selected from the group consisting of straight chain or branched chain alkenyl containing two to ten carbon atoms and substituted straight chain or branched chain alkenyl containing two to ten carbon atoms, wherein the substituent is selected from the group consisting of straight chain or branched chain alkyl containing one to four carbon atoms and cycloalkyl containing three to six carbon atoms; and cycloalkyl containing three to six carbon atoms substituted by straight chain or branched chain alkyl containing one to four carbon atoms; and

R₂₂ is selected from the group consisting of hydrogen, straight chain or branched chain alkyl containing one to eight carbon atoms, benzyl, (phenyl)ethyl and phenyl, the benzyl, (phenyl)ethyl or phenyl substituent being optionally substituted on the benzene ring by one or two moieties independently selected from the group consisting of straight chain or branched chain alkyl containing one to four carbon atoms, straight chain or branched chain alkoxy containing one to four carbon atoms, and halogen, with the proviso that when the benzene ring is substituted by two such moieties, then the moieties together contain no more than six carbon atoms; and

each R₂ is independently selected from the group consisting of straight chain or branched chain alkoxy containing one to four carbon atoms, halogen, and straight chain or branched chain alkyl containing one to four carbon atoms, and n is an integer from zero to 2, with the proviso that if n is 2, then said R₂ groups together contain no more than six carbon atoms;

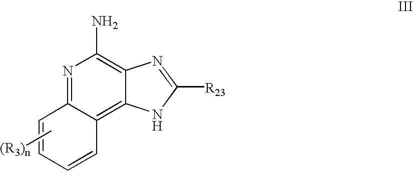

wherein

R₂₃ is selected from the group consisting of hydrogen, straight chain or branched chain alkyl of one to eight carbon atoms, benzyl, (phenyl)ethyl and phenyl, the benzyl, (phenyl)ethyl or phenyl substituent being optionally substituted on the benzene ring by one or two moieties independently selected from the group consisting of straight chain or branched chain alkyl of one to four carbon atoms, straight chain or branched chain alkoxy of one to four carbon atoms, and halogen, with the proviso that when the benzene ring is substituted by two such moieties, then the moieties together contain no more than six carbon atoms; and

each R₃ is independently selected from the group consisting of straight chain or branched chain alkoxy of one to four carbon atoms, halogen, and straight chain or branched chain alkyl of one to four carbon atoms, and n is an integer from zero to 2, with the proviso that if n is 2, then said R₃ groups together contain no more than six carbon atoms;

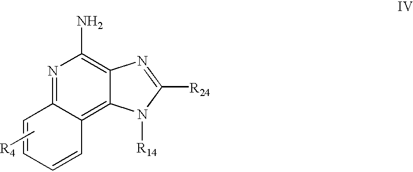

wherein

R₁₄ is —CHRₓR_{y} wherein R_{y} is hydrogen or a carbon-carbon bond, with the proviso that when R_{y} is hydrogen Rₓ is alkoxy of one to four carbon atoms, hydroxyalkoxy of one to four carbon atoms, 1-alkynyl of two to ten carbon atoms, tetrahydropyranyl, alkoxyalkyl wherein the alkoxy moiety contains one to four carbon atoms and the alkyl moiety contains one to four carbon atoms, 2-, 3-, or 4-pyridyl, and with the further proviso that when R_{y} is a carbon-carbon bond R_{y} and Rₓ together form a tetrahydrofuranyl group optionally substituted with one or more substituents independently selected from the group consisting of hydroxy and hydroxyalkyl of one to four carbon atoms;

R₂₄ is selected from the group consisting of hydrogen, alkyl of one to four carbon atoms, phenyl, and substituted phenyl wherein the substituent is selected from the group consisting of alkyl of one to four carbon atoms, alkoxy of one to four carbon atoms, and halogen; and

R₄ is selected from the group consisting of hydrogen, straight chain or branched chain alkoxy containing one to four carbon atoms, halogen, and straight chain or branched chain alkyl containing one to four carbon atoms;

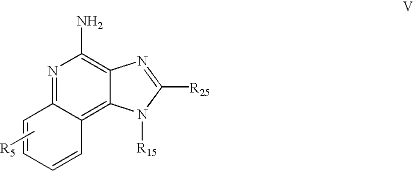

wherein

R₁₅ is selected from the group consisting of: hydrogen; straight chain or branched chain alkyl containing one to ten carbon atoms and substituted straight chain or branched chain alkyl containing one to ten carbon atoms, wherein the substituent is selected from the group consisting of cycloalkyl containing three to six carbon atoms and cycloalkyl containing three to six carbon atoms substituted by straight chain or branched chain alkyl containing one to four carbon atoms; straight chain or branched chain alkenyl containing two to ten carbon atoms and substituted straight chain or branched chain alkenyl containing two to ten carbon atoms, wherein the substituent is selected from the group consisting of cycloalkyl containing three to six carbon atoms and cycloalkyl containing three to six carbon atoms substituted by straight chain or branched chain alkyl containing one to four carbon atoms; hydroxyalkyl of one to six carbon atoms; alkoxyalkyl wherein the alkoxy moiety contains one to four carbon atoms and the alkyl moiety contains one to six carbon atoms; acyloxyalkyl wherein the acyloxy moiety is alkanoyloxy of two to four carbon atoms or benzoyloxy, and the alkyl moiety contains one to six carbon atoms; benzyl; (phenyl)ethyl; and phenyl; said benzyl, (phenyl)ethyl or phenyl substituent being optionally substituted on the benzene ring by one or two moieties independently selected from the group consisting of alkyl of one to four carbon atoms, alkoxy of one to four carbon atoms, and halogen, with the proviso that when said benzene ring is substituted by two of said moieties, then the moieties together contain no more than six carbon atoms;

R₂₅ is

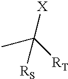

wherein

R_{S} and R_{T} are independently selected from the group consisting of hydrogen, alkyl of one to four carbon atoms, phenyl, and substituted phenyl wherein the substituent is selected from the group consisting of alkyl of one to four carbon atoms, alkoxy of one to four carbon atoms, and halogen;

X is selected from the group consisting of alkoxy containing one to four carbon atoms, alkoxyalkyl wherein the alkoxy moiety contains one to four carbon atoms and the alkyl moiety contains one to four carbon atoms, hydroxyalkyl of one to four carbon atoms, haloalkyl of one to four carbon atoms, alkylamido wherein the alkyl group contains one to four carbon atoms, amino, substituted amino wherein the substituent is alkyl or hydroxyalkyl of one to four carbon atoms, azido, chloro, hydroxy, 1-morpholino, 1-pyrrolidino, alkylthio of one to four carbon atoms; and

R₅ is selected from the group consisting of hydrogen, straight chain or branched chain alkoxy containing one to four carbon atoms, halogen, and straight chain or branched chain alkyl containing one to four carbon atoms; and a pharmaceutically acceptable salt of any of the foregoing.

Suitable 6,7 fused cycloalkylimidazopyridine amine IRM compounds are defined by Formula VI below:

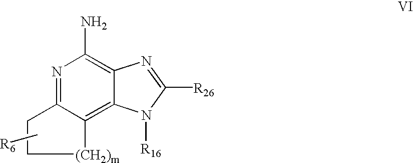

wherein

m is 1, 2, or 3;

R₁₆ is selected from the group consisting of hydrogen; cyclic alkyl of three, four, or five carbon atoms; straight chain or branched chain alkyl containing one to ten carbon atoms and substituted straight chain or branched chain alkyl containing one to ten carbon atoms, wherein the substituent is selected from the group consisting of cycloalkyl containing three to six carbon atoms and cycloalkyl containing three to six carbon atoms substituted by straight chain or branched chain alkyl containing one to four carbon atoms; fluoro- or chloroalkyl containing from one to ten carbon atoms and one or more fluorine or chlorine atoms; straight chain or branched chain alkenyl containing two to ten carbon atoms and substituted straight chain or branched chain alkenyl containing two to ten carbon atoms, wherein the substituent is selected from the group consisting of cycloalkyl containing three to six carbon atoms and cycloalkyl containing three to six carbon atoms substituted by straight chain or branched chain alkyl containing one to four carbon atoms; hydroxyalkyl of one to six carbon atoms; alkoxyalkyl wherein the alkoxy moiety contains one to four carbon atoms and the alkyl moiety contains one to six carbon atoms; acyloxyalkyl wherein the acyloxy moiety is alkanoyloxy of two to four carbon atoms or benzoyloxy, and the alkyl moiety contains one to six carbon atoms, with the proviso that any such alkyl, substituted alkyl, alkenyl, substituted alkenyl, hydroxyalkyl, alkoxyalkyl, or acyloxyalkyl group does not have a fully carbon substituted carbon atom bonded directly to the nitrogen atom; benzyl; (phenyl)ethyl; and phenyl; said benzyl, (phenyl)ethyl or phenyl substituent being optionally substituted on the benzene ring by one or two moieties independently selected from the group consisting of alkyl of one to four carbon atoms, alkoxy of one to four carbon atoms, and halogen, with the proviso that when said benzene ring is substituted by two of said moieties, then the moieties together contain no more than six carbon atoms;

and —CHRₓR_{y}

wherein

R_{y} is hydrogen or a carbon-carbon bond, with the proviso that when R_{y} is hydrogen Rₓ is alkoxy of one to four carbon atoms, hydroxyalkoxy of one to four carbon atoms, 1-alkynyl of two to ten carbon atoms, tetrahydropyranyl, alkoxyalkyl wherein the alkoxy moiety contains one to four carbon atoms and the alkyl moiety contains one to four carbon atoms, 2-, 3-, or 4-pyridyl, and with the further proviso that when R_{y} is a carbon-carbon bond R_{y} and Rₓ together form a tetrahydrofuranyl group optionally substituted with one or more substituents independently selected from the group consisting of hydroxy and hydroxyalkyl of one to four carbon atoms,

R₂₆ is selected from the group consisting of hydrogen, straight chain or branched chain alkyl containing one to eight carbon atoms, straight chain or branched chain hydroxyalkyl containing one to six carbon atoms, morpholinoalkyl, benzyl, (phenyl)ethyl and phenyl, the benzyl, (phenyl)ethyl or phenyl substituent being optionally substituted on the benzene ring by a moiety selected from the group consisting of methyl, methoxy, and halogen; and

—C(R_{S})(R_{T})(X) wherein R_{S} and R_{T} are independently selected from the group consisting of hydrogen, alkyl of one to four carbon atoms, phenyl, and substituted phenyl wherein the substituent is selected from the group consisting of alkyl of one to four carbon atoms, alkoxy of one to four carbon atoms, and halogen;

X is selected from the group consisting of alkoxy containing one to four carbon atoms, alkoxyalkyl wherein the alkoxy moiety contains one to four carbon atoms and the alkyl moiety contains one to four carbon atoms, haloalkyl of one to four carbon atoms, alkylamido wherein the alkyl group contains one to four carbon atoms, amino, substituted amino wherein the substituent is alkyl or hydroxyalkyl of one to four carbon atoms, azido, alkylthio of one to four carbon atoms, and morpholinoalkyl wherein the alkyl moiety contains one to four carbon atoms, and

R₆ is selected from the group consisting of hydrogen, fluoro, chloro, straight chain or branched chain alkyl containing one to four carbon atoms, and straight chain or branched chain fluoro- or chloroalkyl containing one to four carbon atoms and at least one fluorine or chlorine atom;

and pharmaceutically acceptable salts thereof.

Suitable imidazopyridine amine IRM compounds are defined by Formula VII below:

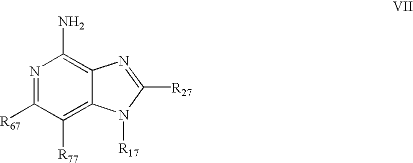

wherein

R₁₇ is selected from the group consisting of hydrogen; —CH₂R_{W} wherein R_{W} is selected from the group consisting of straight chain, branched chain, or cyclic alkyl containing one to ten carbon atoms, straight chain or branched chain alkenyl containing two to ten carbon atoms, straight chain or branched chain hydroxyalkyl containing one to six carbon atoms, alkoxyalkyl wherein the alkoxy moiety contains one to four carbon atoms and the alkyl moiety contains one to six carbon atoms, and phenylethyl; and —CH═CR_{Z}R_{Z} wherein each R_{Z} is independently straight chain, branched chain, or cyclic alkyl of one to six carbon atoms;

R₂₇ is selected from the group consisting of hydrogen, straight chain or branched chain alkyl containing one to eight carbon atoms, straight chain or branched chain hydroxyalkyl containing one to six carbon atoms, alkoxyalkyl wherein the alkoxy moiety contains one to four carbon atoms and the alkyl moiety contains one to six carbon atoms, benzyl, (phenyl)ethyl and phenyl, the benzyl, (phenyl)ethyl or phenyl substituent being optionally substituted on the benzene ring by a moiety selected from the group consisting of methyl, methoxy, and halogen; and morpholinoalkyl wherein the alkyl moiety contains one to four carbon atoms;

R₆₇ and R₇₇ are independently selected from the group consisting of hydrogen and alkyl of one to five carbon atoms, with the proviso that R₆₇ and R₇₇ taken together contain no more than six carbon atoms, and with the further proviso that when R₇₇ is hydrogen then R67 is other than hydrogen and R₂₇ is other than hydrogen or morpholinoalkyl, and with the further proviso that when R67 is hydrogen then R₇₇ and R₂₇ are other than hydrogen;

and pharmaceutically acceptable salts thereof.

Suitable 1,2-bridged imidazoquinoline amine IRM compounds are defined by Formula VIII below:

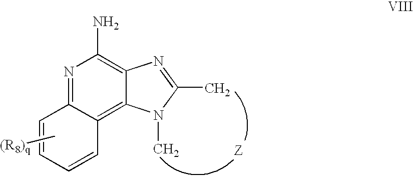

wherein

Z is selected from the group consisting of:

—(CH₂)ₚ— wherein p is 1 to 4;

—(CH₂)ₐ—C(R_{D}R_{E})(CH₂)_{b}—, wherein a and b are integers and a+b is 0 to 3, R_{D} is hydrogen or alkyl of one to four carbon atoms, and R_{E} is selected from the group consisting of alkyl of one to four carbon atoms, hydroxy, —OR_{F} wherein R_{F} is alkyl of one to four carbon atoms, and —NR_{G}R′_{G} wherein R_{G} and R′_{G} are independently hydrogen or alkyl of one to four carbon atoms; and

—(CH₂)ₐ—(Y)—(CH₂)_{b}— wherein a and b are integers and a+b is 0 to 3, and Y is O, S, or —NR_{J}— wherein R_{J} is hydrogen or alkyl of one to four carbon atoms;

q is 0 or 1; and

R₈ is selected from the group consisting of alkyl of one to four carbon atoms, alkoxy of one to four carbon atoms, and halogen, and pharmaceutically acceptable salts thereof.

Suitable thiazolo- and oxazolo-quinolinamine and pyridinamine compounds include compounds defined by Formula IX:

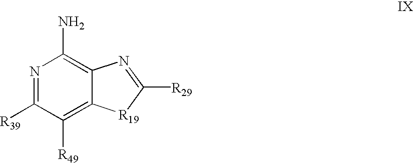

wherein:

R₁₉ is selected from the group consisting of oxygen, sulfur and selenium;

R₂₉ is selected from the group consisting of

-hydrogen;

-alkyl;

-alkyl-OH;

-haloalkyl;

-alkenyl;

-alkyl-X-alkyl;

-alkyl-X-alkenyl;

-alkenyl-X-alkyl;

-alkenyl-X-alkenyl;

-alkyl-N(R₅₉)₂;

-alkyl-N₃;

-alkyl-O—C(O)—N(R₅₉)₂;

-heterocyclyl;

-alkyl-X-heterocyclyl;

-alkenyl-X-heterocyclyl;

-aryl;

-alkyl-X-aryl;

-alkenyl-X-aryl;

-heteroaryl;

-alkyl-X-heteroaryl; and

-alkenyl-X-heteroaryl;

R₃₉ and R₄₉ are each independently:

-hydrogen;

—X-alkyl;

-halo;

-haloalkyl;

—N(R₅₉)₂;

or when taken together, R₃₉ and R₄₉ form a fused aromatic, heteroaromatic, cycloalkyl or heterocyclic ring;

X is selected from the group consisting of —O—, —S—, —NR₅₉—, —C(O)—, —C(O)O—, —OC(O)—, and a bond; and

each R₅₉ is independently H or C₁₋₈alkyl;

and pharmaceutically acceptable salts thereof.

Suitable imidazonaphthyridine and tetrahydroimidazonaphthyridine IRM compounds are those defined by Formulas X and XI below:

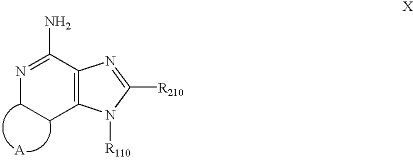

wherein

A is ═N—CR═CR—CR═; ═CR—N═CR—CR═; ═CR—CR═N—CR═; or ═CR—CR═CR—N═;

R₁₁₀ is selected from the group consisting of:

-hydrogen;

—C₁₋₂₀ alkyl or C₂₋₂₀ alkenyl that is unsubstituted or substituted by one or more substituents selected from the group consisting of:

-aryl;

-heteroaryl;

-heterocyclyl;

—O—C₁₋₂₀ alkyl,

—O—(C₁₋₂₀alkyl)₀₋₁-aryl;

—O—(C₁₋₂₀alkyl)₀₋₁-heteroaryl;

—O—(C₁₋₂₀alkyl)₀₋₁-heterocyclyl;

—C₁₋₂₀ alkoxycarbonyl;

—S(O)₀₋₂—C₁₋₂₀ alkyl;

—S(O)₀₋₂—(C₁₋₂₀ alkyl)₀₋₁-aryl;

—S(O)₀₋₂—(C₁₋₂₀ alkyl)₀₋₁-heteroaryl;

—S(O)₀₋₂—(C₁₋₂₀ alkyl)₀₋₁-heterocyclyl;

-N(R₃₁₀)₂;

—N₃;

oxo;

-halogen;

—NO₂;

—OH; and

—SH; and

—C₁₋₂₀ alkyl-NR₃₁₀-Q-X—R₄₁₀ or —C₂₋₂₀ alkenyl-NR₃₁₀-Q-X—R₄₁₀ wherein Q is —CO— or —SO₂—; X is a bond, —O— or —NR₃₁₀- and R₄₁₀ is aryl; heteroaryl; heterocyclyl; or —C₁₋₂₀ alkyl or C₂₋₂₀ alkenyl that is unsubstituted or substituted by one or more substituents selected from the group consisting of:

-aryl;

-heteroaryl;

-heterocyclyl;

—O—C₁₋₂₀ alkyl,

—O—(C₁₋₂₀alkyl)₀₋₁-aryl;

—O—(C₁₋₂₀alkyl)₀₋₁-heteroaryl;

—O—(C₁₋₂₀alkyl)₀₋₁-heterocyclyl;

—C₁₋₂₀ alkoxycarbonyl;

—S(O)₀₋₂—C₁₋₂₀ alkyl;

—S(O)₀₋₂—(C₁₋₂₀ alkyl)₀₋₁-aryl;

—S(O)₀₋₂—(C₁₋₂₀ alkyl)₀₋₁-heteroaryl;

—S(O)₀₋₂—(C₁₋₂₀ alkyl)₀₋₁-heterocyclyl;

—N(R₃₁₀)₂;

—NR₃₁₀—CO—O—C₁₋₂₀alkyl;

—N₃;

oxo;

-halogen;

—NO₂;

—OH; and

—SH; or R₄₁₀ is

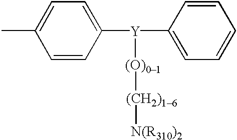

wherein Y is —N— or —CR—;

R₂₁₀ is selected from the group consisting of:

-hydrogen;

—C₁₋₁₀ alkyl;

—C₂₋₁₀ alkenyl;

-aryl;

—C₁₋₁₀ alkyl-O-C₁₋₁₀ alkyl;

—C₁₋₁₀ alkyl-O—C₂₋₁₀ alkenyl; and

—C₁₋₁₀ alkyl or C₂₋₁₀ alkenyl substituted by one or more substituents selected from the group consisting of:

—OH;

-halogen;

—N(R₃₁₀)₂;

—CO—N(R₃₁₀)₂;

—CO—C₁₋₁₀ alkyl;

—N₃;

-aryl;

-heteroaryl;

-heterocyclyl;

—CO-aryl; and

—CO-heteroaryl;

each R₃₁₀ is independently selected from the group consisting of hydrogen and C₁₋₁₀ alkyl; and

each R is independently selected from the group consisting of hydrogen, C₁₋₁₀ alkyl, C₁₋₁₀ alkoxy, halogen and trifluoromethyl,

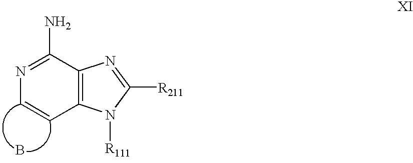

wherein

B is —NR—C(R)₂—C(R)₂—C(R)₂—; —C(R)₂—NR—C(R)₂—C(R)₂—; —C(R)₂—C(R)₂—NR—C(R)₂— or —C(R)₂—C(R)₂—C(R)₂—NR—;

R₁₁₁ is selected from the group consisting of:

-hydrogen;

—C₁₋₂₀ alkyl or C₂₋₂₀ alkenyl that is unsubstituted or substituted by one or more substituents selected from the group consisting of:

-aryl;

-heteroaryl;

-heterocyclyl;

—O—C₁₋₂₀ alkyl;

—O—(C₁₋₂₀alkyl)₀₋₁-aryl;

—O-(C₁₋₂₀alkyl)₀₋₁-heteroaryl;

—O—(C₁₋₂₀alkyl)₀₋₁-heterocyclyl;

-C₁₋₂₀ alkoxycarbonyl;

—S(O)₀₋₂—C₁₋₂₀ alkyl;

—S(O)₀₋₂—(C₁₋₂₀ alkyl)₀₋₁-aryl;

—S(O)₀₋₂—(C₁₋₂₀ alkyl)₀₋₁-heteroaryl;

—S(O)₀₋₂—(C₁₋₂₀ alkyl)₀₋₁-heterocyclyl;

—N(R₃₁₁)₂;

—N₃;

oxo;

-halogen;

—NO₂;

—OH; and

—SH; and

—C₁₋₂₀ alkyl-NR₃₁₁-Q-X—R₄₁₁ or —C₂₋₂₀ alkenyl-NR₃₁₁-Q-X—R₄₁₁ wherein Q is —CO— or —SO₂—; X is a bond, —O— or —NR₃₁₁— and R₄₁₁ is aryl; heteroaryl; heterocyclyl; or —C₁₋₂₀ alkyl or C₂₋₂₀ alkenyl that is unsubstituted or substituted by one or more substituents selected from the group consisting of:

-aryl;

-heteroaryl;

-heterocyclyl;

—O—C₁₋₂₀alkyl,

—O—(C₁₋₂₀alkyl)₀₋₁-aryl;

—O—(C₁₋₂₀alkyl)₀₋₁-heteroaryl;

—O-(C₁₋₂₀alkyl)₀₋₁-heterocyclyl;

-C₁₋₂₀ alkoxycarbonyl;

—S(O)₀₋₂—C₁₋₂₀ alkyl;

—S(O)₀₋₂—(C₁₋₂₀ alkyl)₀₋₁-aryl;

—S(O)₀₋₂—(C₁₋₂₀ alkyl)₀₋₁-heteroaryl;

—S(O)₀₋₂—(C₁₋₂₀ alkyl)₀₋₁-heterocyclyl;

—N(R₃₁₁)₂;

—NR₃₁₁—CO—O—C₁₋₂₀alkyl;

—N₃;

oxo;

-halogen;

—NO₂;

—OH; and

—SH; or R₄₁₁ is

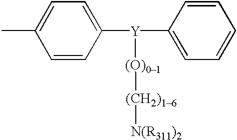

wherein Y is —N— or —CR—;

R₂₁₁ is selected from the group consisting of:

-hydrogen;

—C₁₋₁₀ alkyl;

—C₂₋₁₀ alkenyl;

-aryl

—C₁₋₁₀ alkyl —O—C₁₋₁₀-alkyl;

—C₁₋₁₀ alkyl-O—C₂₋₁₀ alkenyl; and

—C₁₋₁₀ alkyl or C₂₋₁₀ alkenyl substituted by one or more substituents selected from the group consisting of:

—OH;

-halogen;

—N(R₃₁₁)₂;

—CO—N(R₃₁₁)₂;

—CO-C₁₋₁₀ alkyl;

—N₃;

-aryl;

-heteroaryl;

-heterocyclyl;

-CO-aryl; and

—CO-heteroaryl;

each R₃₁₁ is independently selected from the group consisting of hydrogen and C₁₋₁₀ alkyl; and

each R is independently selected from the group consisting of hydrogen, C₁₋₁₀ alkyl, C₁₋₁₀ alkoxy, halogen and trifluoromethyl, and pharmaceutically acceptable salts thereof.

Additional suitable 1H-imidazo[4,5-c]quinolin-4-amines and tetrahydro-1H-imidazo[4,5-c]quinolin-4-amines include compounds defined by Formulas XII, XIII and XIV below:

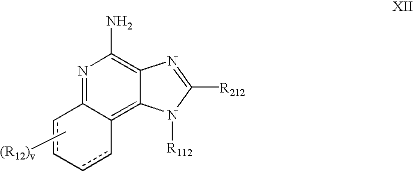

wherein

R₁₁₂ is -alkyl-NR₃₁₂—CO—R₄₁₂ or -alkenyl-NR₃₁₂—CO—R₄₁₂ wherein R₄₁₂ is aryl, heteroaryl, alkyl or alkenyl, each of which may be unsubstituted or substituted by one or more substituents selected from the group consisting of:

-alkyl;

-alkenyl;

-alkynyl;

-(alkyl)₀₋₁-aryl;

-(alkyl)₀₋₁-(substituted aryl);

-(alkyl)₀₋₁-heteroaryl;

-(alkyl)₀₋₁-(substituted heteroaryl);

—O-alkyl;

—O-(alkyl)₀₋₁-aryl;

-O-(alkyl)₀₋₁-(substituted aryl);

—O-(alkyl)₀₋₁-heteroaryl;

—O-(alkyl)₀₋₁-(substituted heteroaryl);

—CO-aryl;

—CO-(substituted aryl);

—CO-heteroaryl;

—CO-(substituted heteroaryl);

—COOH;

—CO—O-alkyl;

—CO-alkyl;

—S(O)₀₋₂-alkyl;

—S(O)₀₋₂-(alkyl)₀₋₁-aryl;

—S(O)₀₋₂-(alkyl)₀₋₁-(substituted aryl);

—S(O)₀₋₂-(alkyl)₀₋₁-heteroaryl;

—S(O)₀₋₂-(alkyl)₀₋₁-(substituted heteroaryl);

—P(O)(OR₃₁₂)₂;

—NR₃₁₂—CO—O-alkyl;

—N₃;

-halogen;

—NO₂;

—CN;

-haloalkyl;

—O-haloalkyl;

—CO-haloalkyl;

—OH;

—SH; and in the case of alkyl, alkenyl, or heterocyclyl, oxo;

or R₄₁₂ is

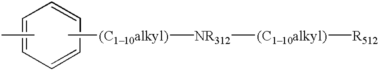

wherein R₅₁₂ is an aryl, (substituted aryl), heteroaryl, (substituted heteroaryl), beterocyclyl or (substituted heterocyclyl) group;

R₂₁₂ is selected from the group consisting of:

-hydrogen;

-alkyl;

-alkenyl;

-aryl;

-(substituted aryl);

-heteroaryl;

-(substituted heteroaryl);

-heterocyclyl;

-(substituted heterocyclyl);

-alkyl-O-alkyl;

-alkyl-O-alkenyl; and

-alkyl or alkenyl substituted by one or more substituents selected from the group consisting of:

—OH;

-halogen;

—N(R₃₁₂)₂;

—CO—N(R₃₁₂)₂;

—CO—C₁₋₁₀ alkyl;

—CO—O—C₁₋₁₀ alkyl;

—N₃;

-aryl;

-(substituted aryl);

-heteroaryl;

-(substituted heteroaryl);

-heterocyclyl;

-(substituted heterocyclyl);

—CO-aryl; and

—CO-heteroaryl;

each R₃₁₂ is independently selected from the group consisting of hydrogen; C₁₋₁₀ alkyl-heteroaryl; C₁₋₁₀ alkyl-(substituted heteroaryl); C₁₋₁₀ alkyl-aryl; C₁₋₁₀ alkyl-(substituted aryl) and C₁₋₁₀ alkyl;

v is 0 to 4;

and each R₁₂ present is independently selected from the group consisting of C₁₋₁₀ alkyl, C₁₋₁₀ alkoxy, halogen and trifluoromethyl;

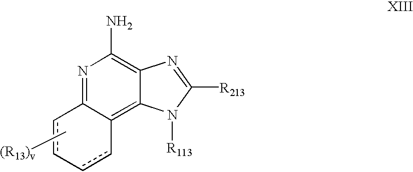

wherein

R₁₁₃ is -alkyl-NR₃₁₃—SO₂—X—R₄₁₃ or -alkenyl-NR₃₁₃—SO₂—X—R₄₁₃;

X is a bond or —NR₅₁₃—;

R₄₁₃ is aryl, heteroaryl, heterocyclyl, alkyl or alkenyl, each of which may be unsubstituted or substituted by one or more substituents selected from the group consisting of:

-alkyl;

-alkenyl;

-aryl;

-heteroaryl;

-heterocyclyl;

-substituted cycloalkyl;

-substituted aryl;

-substituted heteroaryl;

-substituted heterocyclyl;

-O-alkyl;

—O-(alkyl)₀₋₁-aryl;

—O-(alkyl)₀₋₁-substituted aryl;

—O-(alkyl)₀₋₁-heteroaryl;

—O-(alkyl)₀₋₁-substituted heteroaryl;

—O-(alkyl)₀₋₁-heterocyclyl;

—O-(alkyl)₀₋₁-substituted heterocyclyl;

—COOH;

—CO—O-alkyl;

—CO-alkyl;

—S(O)₀₋₂-alkyl;

—S(O)₀₋₂-(alkyl)₀₋₁-aryl;

—S(O)₀₋₂-(alkyl)₀₋₁-substituted aryl;

—S(O)₀₋₂-(alkyl)₀₋₁-heteroaryl;

—S(O)₀₋₂-(alkyl)₀₋₁-substituted heteroaryl;

—S(O)₀₋₂-(alkyl)₀₋₁-heterocyclyl;

—S(O)₀₋₂-(alkyl)₀₋₁-substituted heterocyclyl;

-(alkyl)₀₋₁-NR₃₁₃R₃₁₃;

-(alkyl)₀₋₁-NR₃₁₃—CO—O-alkyl;

-(alkyl)₀₋₁-NR₃₁₃—CO-alkyl;

-(alkyl)₀₋₁-NR₃₁₃—CO-aryl;

-(alkyl)₀₋₁-NR₃₁₃—CO-substituted aryl;

-(alkyl)₀₋₁-NR₃₁₃—CO-heteroaryl;

-(alkyl)₀₋₁-NR₃₁₃—CO-substituted heteroaryl;

—N₃;

-halogen;

-haloalkyl;

-haloalkoxy;

—CO-haloalkyl;

—CO-haloalkoxy;

—NO₂;

—CN;

—OH;

—SH; and in the case of alkyl, alkenyl, or heterocyclyl, oxo;

R₂₁₃ is selected from the group consisting of:

-hydrogen;

-alkyl;

-alkenyl;

-aryl;

-substituted aryl;

-heteroaryl;

-substituted heteroaryl;

-alkyl-O-alkyl;

-alkyl-O-alkenyl; and

-alkyl or alkenyl substituted by one or more substituents selected from the group consisting of:

—OH;

-halogen;

—N(R₃₁₃)₂;

—CO—N(R₃₁₃)₂;

—CO—C₁₋₁₀ alkyl;

—CO—O—C₁₋₁₀ alkyl;

—N₃;

-aryl;

-substituted aryl;

-heteroaryl;

-substituted heteroaryl;

-heterocyclyl;

-substituted heterocyclyl;

—CO-aryl;

—CO-(substituted aryl);

—CO-heteroaryl; and

—CO-(substituted heteroaryl);

each R₃₁₃ is independently selected from the group consisting of hydrogen and C₁₋₁₀ alkyl;

R₅₁₃ is selected from the group consisting of hydrogen and C₁₋₁₀ alkyl, or R₄₁₃ and R₅₁₃ can combine to form a 3 to 7 membered heterocyclic or substituted heterocyclic ring;

v is 0 to 4;

and each R₁₃ present is independently selected from the group consisting of C₁₋₁₀ alkyl, C₁₋₁₀ alkoxy, halogen and trifluoromethyl;

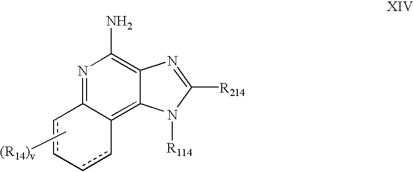

wherein

R₁₁₄ is -alkyl-NR₃₁₄—CY—NR₅₁₄—X—R₄₁₄ or

-alkenyl-NR₃₁₄—CY—NR₅₁₄—X—R₄₁₄

wherein

Y is ═O or ═S;

X is a bond, —CO— or —SO₂—;

R₄₁₄ is aryl, heteroaryl, heterocyclyl, alkyl or alkenyl, each of which may be unsubstituted or substituted by one or more substituents selected from the group consisting of:

-alkyl;

-alkenyl;

-aryl;

-heteroaryl;

-heterocyclyl;

-substituted aryl;

-substituted heteroaryl;

-substituted heterocyclyl;

-O-alkyl;

—O-(alkyl)₀₋₁-aryl;

—O-(alkyl)₀₋₁-substituted aryl;

—O-(alkyl)₀₋₁-heteroaryl;

—O-(alkyl)₀₋₁-substituted heteroaryl;

—O-(alkyl)₀₋₁-heterocyclyl;

-O-(alkyl)₀₋₁-substituted heterocyclyl;

—COOH;

—CO—O-alkyl;

—CO-alkyl;

—S(O)₀₋₂-alkyl;

—S(O)₀₋₂-alkyl)₀₋₁-aryl;

—S(O)₀₋₂-alkyl)₀₋₁-substituted aryl;

—S(O)₀₋₂-(alkyl)₀₋₁-heteroaryl;

—S(O)₀₋₂-(alkyl)₀₋₁-substituted heteroaryl;

—S(O)₀₋₂-(alkyl)₀₋₁-heterocyclyl;

—S(O)₀₋₂-(alkyl)₀₋₁-substituted heterocyclyl;

-(alkyl)₀₋₁-NR₃₁₄R₃₁₄;

-(alkyl)₀₋₁-NR₃₁₄—CO—O-alkyl;

-(alkyl)₀₋₁-NR₃₁₄—CO-alkyl;

-(alkyl)₀₋₁-NR₃₁₄—CO-aryl;

-(alkyl)₀₋₁-NR₃₁₄—CO-substituted aryl;

-(alkyl)₀₋₁-NR₃₁₄—CO-heteroaryl;

-(alkyl)₀₋₁-NR₃₁₄—CO-substituted heteroaryl;

—N₃;

-halogen;

-haloalkyl;

-haloalkoxy;

—CO-haloalkoxy;

—NO₂;

—CN;

—OH;

—SH; and, in the case of alkyl, alkenyl or heterocyclyl, oxo;

with the proviso that when X is a bond R₄₁₄ can additionally be hydrogen;

R₂₁₄ is selected from the group consisting of:

-hydrogen;

-alkyl;

-alkenyl;

-aryl;

-substituted aryl;

-heteroaryl;

-substituted heteroaryl;

-alkyl-O-alkyl;

-alkyl-O-alkenyl; and

-alkyl or alkenyl substituted by one or more substituents selected from the group consisting of:

—OH;

-halogen;

—N(R₃₁₄)₂;

—CO—N(R₃₁₄)₂;

—CO—C₁₋₁₀ alkyl;

—CO—O—C₁₋₁₀ alkyl;

—N₃;

-aryl;

-substituted aryl;

-heteroaryl;

-substituted heteroaryl;

-heterocyclyl;

-substituted heterocyclyl;

—CO-aryl;

—CO-(substituted aryl);

—CO-heteroaryl; and

—CO-(substituted heteroaryl);

each R₃₁₄ is independently selected from the group consisting of hydrogen and C₁₋₁₀ alkyl;

R₅₁₄ is selected from the group consisting of hydrogen and C₁₋₁₀ alkyl, or R₄₁₄ and R₅₁₄ can combine to form a 3 to 7 membered heterocyclic or substituted heterocyclic ring;

v is 0 to 4;

and each R₁₄ present is independently selected from the group consisting of C₁₋₁₀ alkyl, C₁₋₁₀ alkoxy, halogen and trifluoromethyl,

and pharmaceutically acceptable salts thereof.

Additional suitable 1H-imidazo[4,5-c]quinolin-4-amines and tetrahydro-1H-imidazo[4,5-c]quinolin-4-amines include compounds defined by Formulas XV, XVI, XVII, XVIII, XIX, XX, XXI, XXII, XXIII, XXIV, XXV, and XXVI below

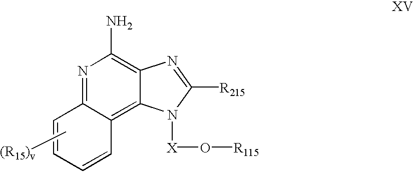

wherein:

X is —CHR₅₁₅—, —CHR₅₁₅-alkyl-, or —CHR₅₁₅-alkenyl-;

R₁₁₅ is selected from the group consisting of:

—R₄₁₅—CR₃₁₅—Z—R₆₁₅-alkyl;

—R₄₁₅—CR₃₁₅-Z-R₆₁₅-alkenyl;

—R₄₁₅—CR₃₁₅-Z-R₆₁₅-aryl;

—R₄₁₅—CR₃₁₅-Z-R₆₁₅-heteroaryl;

—R₄₁₅—CR₃₁₅-Z-R₆₁₅-heterocyclyl;

—R₄₁₅—CR₃₁₅Z-H;

—R₄₁₅-NR₇₁₅—CR₃₁₅—R₆₁₅-alkyl;

—R₄₁₅-NR₇₁₅—CR₃₁₅—R₆₁₅-alkenyl;

—R₄₁₅-NR₇₁₅—CR₃₁₅—R₆₁₅-aryl;

—R₄₁₅—NR₇₁₅—CR₃₁₅—R₆₁₅-heteroaryl;

—R₄₁₅—NR₇₁₅—CR₃₁₅—R₆₁₅-heterocyclyl; and

—R₄₁₅—NR₇₁₅—CR₃₁₅—R₈₁₅;

Z is —NR₅₁₅—, —O—, or —S—;

R₂₁₅ is selected from the group consisting of:

-hydrogen;

-alkyl;

-alkenyl;

-aryl;

-heteroaryl;

-heterocyclyl;

-alkyl-Y-alkyl;

-alkyl-Y-alkenyl;

-alkyl-Y-aryl; and

-alkyl or alkenyl substituted by one or more substituents selected from the group consisting of:

—OH;

-halogen;

—N(R₅₁₅)₂;

—CO—N(R₅₁₅)₂;

—CO—C₁₋₁₀ alkyl;

—CO—O—C₁₋₁₀ alkyl;

—N₃;

-aryl;

-heteroaryl;

-heterocyclyl;

—CO-aryl; and

—CO-heteroaryl;

R₃₁₅ is ═O or ═S;

R₄₁₅ is alkyl or alkenyl, which may be interrupted by one or more —O— groups;

each R₅₁₅ is independently H or C₁₋₁₀ alkyl;

R₆₁₅ is a bond, alkyl, or alkenyl, which may be interrupted by one or more —O— groups;

R₇₁₅ is H, C₁₋₁₀ alkyl, or arylalkyl; or R₄₁₅ and R₇₁₅ can join together to form a ring;

R₈₁₅ is H or C₁₋₁₀ alkyl; or R₇₁₅ and R₈₁₅ can join together to form a ring;

Y is −O— or —S(O)₀₋₂—;

v is 0 to 4; and

each R₁₅ present is independently selected from the group consisting of C₁₋₁₀ alkyl, C₁₋₁₀ alkoxy, hydroxy, halogen and trifluoromethyl;

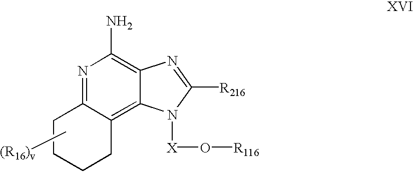

wherein:

X is —CHR₅₁₆—, —CHR₅₁₆-alkyl-, or —CHR₅₁₆-alkenyl-;

R₁₁₆ is selected from the group consisting of:

—R₄₁₆—CR₃₁₆-Z-R₆₁₆-alkyl;

—R₄₁₆—CR₃₁₆-Z-R₆₁₆-alkenyl;

—R₄₁₆-CR₃₁₆-Z-R₆₁₆-aryl;

—R₄₁₆-CR₃₁₆-Z-R₆₁₆-heteroaryl;

—R₄₁₆-CR₃₁₆-Z-R616-heterocyclyl;

—R₄₁₆—CR₃₁₆-Z-H;

—R₄₁₆-NR₇₁₆—CR₃₁₆—R₆₁₆alkyl;

—R₄₁₆—NR₇₁₆—CR₃₆—R₆₁₆alkenyl;

—R₄₁₆—NR₇₁₆—CR₃₁₆—R₆₁₆-aryl;

—R₄₁₆—NR₇₁₆—CR₃₁₆—R₆₁₆-heteroaryl;

—R₄₁₆—NR₇₁₆—CR₃₁₆—R₆₁₆-heterocyclyl; and

—R₄₁₆—NR₇₁₆—CR₃₁₆—R₈₁₆;

Z is —NR₅₁₆—, —O—, or —S—;

R₂₁₆ is selected from the group consisting of:

-hydrogen;

-alkyl;

-alkenyl;

-aryl;

-heteroaryl;

-heterocyclyl;

-alkyl-Y-alkyl;

-alkyl-Y-lkenyl;

-alkyl-Y-aryl; and

-alkyl or alkenyl substituted by one or more substituents selected from the group consisting of:

—OH;

-halogen;

—N(R₅₁₆)₂;

—CO—N(R₅₁₆)₂;

—CO—C₁₋₁₀ alkyl;

—CO—O—C₁₋₁₀ alkyl;

—N₃;

-aryl;

-heteroaryl;

-heterocyclyl;

—CO-aryl; and

—CO-heteroaryl;

R₃₁₆ is ═O or ═S;

R₄₁₆ is alkyl or alkenyl, which may be interrupted by one or more —O— groups;

each R₅₁₆ is independently H or C₁₋₁₀ alkyl;

R₆₁₆ is a bond, alkyl, or alkenyl, which may be interrupted by one or more —O— groups;

R₇₁₆ is H, C₁₋₁₀ alkyl, arylalkyl; or R₄₁₆ and R₇₁₆ can join together to form a ring;

R₈₁₆ is H or C₁₋₁₀ alkyl; or R₇₁₆ and R₈₁₆ can join together to form a ring;

Y is —O— or —S(O)₀₋₂;

v is 0 to 4; and

each R₁₆ present is independently selected from the group consisting of C₁₋₁₀ alkyl, C₁₋₁₀ alkoxy, hydroxy, halogen, and trifluoromethyl;

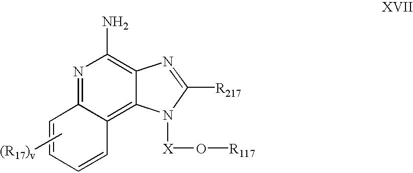

wherein:

X is —CHR₃₁₇—, —CHR₃₁₇-alkyl-, or —CHR₃₁₇-alkenyl-;

R₁₁₇ is selected from the group consisting of:

-alkenyl;

-aryl; and

—R₄₁₇ aryl;

R₂₁₇ is selected from the group consisting of:

-hydrogen;

-alkyl;

-alkenyl;

-aryl;

-heteroaryl;

-heterocyclyl;

-alkyl-Y-alkyl;

-alkyl-Y-alkenyl;

-alkyl-Y-aryl; and

-alkyl or alkenyl substituted by one or more substituents selected from the group consisting of:

—OH;

-halogen;

—N(R₃₁₇)₂;

—CO—N(R₃₁₇)₂;

—CO—C₁₋₁₀ alkyl;

—CO—O—C₁₋₁₀ alkyl;

—N₃;

-aryl;

-heteroaryl;

-heterocyclyl;

—CO-aryl; and

—CO-heteroaryl;

R₄₁₇ is alkyl or alkenyl, which may be interrupted by one or more —O— groups;

each R₃₁₇ is independently H or C₁₋₁₀ alkyl;

each Y is independently —O— or —S(O)₀₋₂—;

v is 0 to 4; and

each R₁₇ present is independently selected from the group consisting of C₁₋₁₀ alkyl, C₁₋₁₀ alkoxy, hydroxy, halogen and trifluoromethyl;

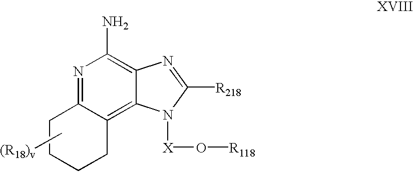

wherein:

X is —CHR₃₁₈—, —CHR₃,₈-alkyl-, or —CHR₃,₈-alkenyl-;

R₁₁₈ is selected from the group consisting of:

-aryl;

-alkenyl; and

—R₄₁₈-aryl;

R₂₁₈ is selected from the group consisting of:

-hydrogen;

-alkyl;

-alkenyl;

-aryl;

-heteroaryl;

-heterocyclyl;

-alkyl-Y-alkyl;

-alkyl-Y-aryl;

-alkyl-Y-alkenyl; and

-alkyl or alkenyl substituted by one or more substituents selected from the group consisting of:

—OH;

-halogen;

—N(R₃₁₈)₂;

—CO—N(R₃₁₈)₂;

—CO—C₁₋₁₀ alkyl;

—CO—O—C₁₋₁₀ alkyl;

—N₃;

-aryl;

-heteroaryl;

-heterocyclyl;

—CO-aryl; and

—CO-heteroaryl;

R₄₁₈ is alkyl or alkenyl, which may be interrupted by one or more —O— groups;

each R₃₁₈ is independently H or C₁₋₁₀ alkyl;

each Y is independently —O— or —S(O)₀₋₂—;

v is 0 to 4; and

each R₁₈ present is independently selected from the group consisting of C₁₋₁₀ alkyl, C₁₋₁₀ alkoxy, hydroxy, halogen and trifluoromethyl;

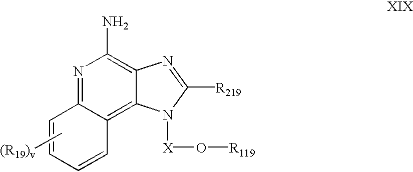

wherein:

X is —CHR₃₁₉—, —CHR₃₁₉-alkyl-, or —CHR₃I₉-alkenyl-;

R₁₁₉ is selected from the group consisting of:

-heteroaryl;

-heterocyclyl;

—R₄₁₉-heteroaryl; and

—R₄₁₉-heterocyclyl;

R₂₁₉ is selected from the group consisting of:

-hydrogen;

-alkyl;

-alkenyl;

-aryl;

-heteroaryl;

-heterocyclyl;

-alkyl-Y-alkyl;

-alkyl-Y-alkenyl;

-alkyl-Y-aryl; and

-alkyl or alkenyl substituted by one or more substituents selected from the group consisting of:

—OH;

-halogen;

—N(R₃₁₉)₂;

—CO—N(R₃₁₉)₂;

—CO—C₁₋₁₀ alkyl;

—CO—O—C₁₋₁₀ alkyl;

—N₃;

-aryl;

-heteroaryl;

-heterocyclyl;

—CO-aryl; and

—CO-heteroaryl;

R₄₁₉ is alkyl or alkenyl, which may be interrupted by one or more —O— groups;

each R₃₁₉ is independently H or C₁₋₁₀ alkyl;

each Y is independently —O— or —S(O)₀₋₂—;

v is 0 to 4; and

each R₁₉ present is independently selected from the group consisting of C₁₋₁₀ alkyl, C₁₋₁₀ alkoxy, hydroxy, halogen and trifluoromethyl;

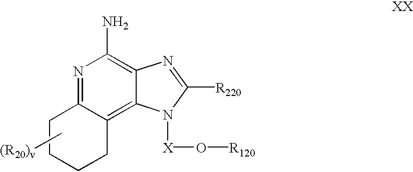

wherein:

X is —CHR₃₂₀—, —CHR₃₂₀-alkyl-, or —CHR₃₂₀-alkenyl-;

R₁₂₀ is selected from the group consisting of:

-heteroaryl;

-heterocyclyl;

—R₄₂₀-heteroaryl; and

—R₄₂₀-heterocyclyl;

R₂₂₀ is selected from the group consisting of:

-hydrogen;

-alkyl;

-alkenyl;

-aryl;

-heteroaryl;

-heterocyclyl;

-alkyl-Y-alkyl;

-alkyl-Y-alkenyl;

-alkyl-Y-aryl; and

-alkyl or alkenyl substituted by one or more substituents selected from the group consisting of:

—OH;

-halogen;

—N(R₃₂₀)₂;

—CO—N(R₃₂₀)₂;

—CO—C₁₋₁₀ alkyl;

—CO—O—C₁₋₁₀ alkyl;

—N₃;

-aryl;

-heteroaryl;

-heterocyclyl;

—CO-aryl; and

—CO-heteroaryl;

R₄₂₀ is alkyl or alkenyl, which may be interrupted by one or more —O— groups;

each R₃₂₀ is independently H or C₁₋₁₀ alkyl;

each Y is independently —O— or —S(O)₀₋₂—;

v is 0 to 4; and

each R₂₀ present is independently selected from the group consisting of C₁₋₁₀ alkyl, C₁₋₁₀ alkoxy, hydroxy, halogen and trifluoromethyl;

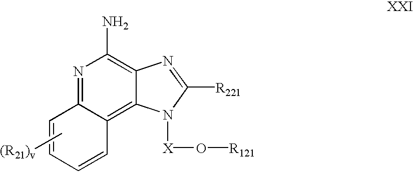

wherein:

X is —CHR₅₂₁—, —CHR₅₂₁-alkyl-, or —CHR₅₂1-alkenyl-;

R₁₂₁ is selected from the group consisting of:

—R₄₂₁—NR₃₂₁—SO₂—R₆₂₁-alkyl;

—R₄₂₁—NR₃₂₁—SO₂—R₆₂₁-alkenyl;

—R₄₂₁—NR₃₂₁—SO₂—R₆₂₁-aryl;

—R₄₂₁—NR₃₂₁—SO₂—R₆₂₁-heteroaryl;

—R₄₂₁—NR₃₂₁—SO₂—R₆₂₁-heterocyclyl;

—R₄₂₁—NR₃₂₁—SO₂—R₇₂₁;

—R₄₂₁—NR₃₂₁—SO₂—NR₅₂₁—R₆₂₁-alkyl;

—R₄₂₁—NR₃₂₁—SO₂—NR₅₂₁—R₆₂₁-alkenyl;

—R₄₂₁—NR₃₂₁—SO₂—NR₅₂₁—R₆₂₁-aryl;

—R₄₂₁—NR₃₂₁—SO₂—NR₅₂₁—R₆₂₁-heteroaryl;

—R₄₂₁—NR₃₂₁—SO₂—NR₅₂₁—R₆₂₁-heterocyclyl; and

—R₄₂₁—NR₃₂₁—SO₂—NH₂;

R₂₂₁ is selected from the group consisting of:

-hydrogen;

-alkyl;

-alkenyl;

-aryl;

-heteroaryl;

-heterocyclyl;

-alkyl-Y-alkyl;

-alkyl-Y-alkenyl;

-alkyl-Y-aryl; and

-alkyl or alkenyl substituted by one or more substituents selected from the group consisting of:

—OH;

-halogen;

—N(R₅₂₁)₂;

—CO—N(R₅₂₁)₂;

—CO—C₁₋₁₀ alkyl;

—CO—O—C₁₋₁₀ alkyl;

—N₃;

-aryl;

-heteroaryl;

-heterocyclyl;

—CO-aryl; and

—CO-heteroaryl;

Y is —O— or —S(O)₀₋₂—;

R₃₂₁ is H, C₁₋₁₀ alkyl, or arylalkyl;

each R₄₂₁ is independently alkyl or alkenyl, which may be interrupted by one or more —O— groups; or R₃₂₁ and R₄₂₁ can join together to form a ring;

each R₅₂₁ is independently H, C₁₋₁₀ alkyl, or C₂₋₁₀ alkenyl;

R₆₂₁ is a bond, alkyl, or alkenyl, which may be interrupted by one or more —O— groups;

R₇₂₁ is C₁₋₁₀ alkyl; or R₃₂₁ and R₇₂₁ can join together to form a ring;

v is 0 to 4; and

each R₂₁ present is independently selected from the group consisting of C₁₋₁₀ alkyl, C₁₋₁₀ alkoxy, hydroxy, halogen and trifluoromethyl;

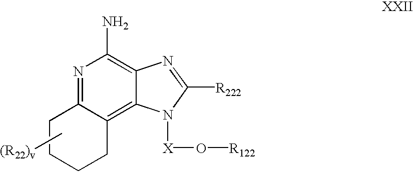

wherein:

X is —CHR₅₂₂—, —CHR₅₂₂-alkyl-, or —CHR₅₂₂-alkenyl-;

R₁₂₂ is selected from the group consisting of:

—R₄₂₂—NR₃₂₂—SO₂—R₆₂₂-alkyl;

—R₄₂₂—NR₃₂₂—SO₂—R₆₂₂-alkenyl;

—R₄₂₂—NR₃₂₂—SO₂—R₆₂₂-aryl;

—R₄₂₂—NR₃₂₂—SO₂—R₆₂₂-heteroaryl;

—R₄₂₂—NR₃₂₂—SO₂—R₆₂₂-heterocyclyl;

—R₄₂₂—NR₃₂₂—SO₂—R₇₂₂;

—R₄₂₂—NR₃₂₂—SO₂—NR₅₂₂-R₆₂₂-alkyl;

—R₄₂₂—NR₃₂₂—SO₂—NR₅₂₂—R₆₂₂-alkenyl;

—R₄₂₂—NR₃₂₂—SO₂—NR₅₂₂—R₆₂₂-aryl;

—R₄₂₂—NR₃₂₂—SO₂—NR₅₂₂—R₆₂₂-heteroaryl;

—R₄₂₂—NR₃₂₂—SO₂—NR₅₂₂—R₆₂₂-heterocyclyl; and

—R₄₂₂—NR₃₂₂—SO₂—NH₂;

R₂₂₂ is selected from the group consisting of:

-hydrogen;

-alkyl;

-alkenyl;

-aryl;

-heteroaryl;

-heterocyclyl;

-alkyl-Y-alkyl;

-alkyl-Y-alkenyl;

-alkyl-Y-aryl; and

-alkyl or alkenyl substituted by one or more substituents selected from the group consisting of:

—OH;

-halogen;

—N(R₅₂₂)₂;

—CO—N(R₅₂₂)₂;

—CO-C₁₋₁₀ alkyl;

—CO—O—C₁₋₁₀ alkyl;

—N₃;

-aryl;

-heteroaryl;

-heterocyclyl;

—CO-aryl; and

—CO-heteroaryl;

Y is —O— or —S(O)₀₋₂—;

R₃₂₂ is H, C₁₋₁₀ alkyl, or arylalkyl;

each R₄₂₂ is independently alkyl or alkenyl, which may be interrupted by one or more —O— groups; or R₃₂₂ and R₄₂₂ can join together to form a ring;

each R₅₂₂ is independently H, C₁₋₁₀ alkyl, or C₂₋₁₀ alkenyl;

R₆₂₂ is a bond, alkyl, or alkenyl, which may be interrupted by one or more —O— groups;

R₇₂₂ is C₁₋₁₀ alkyl; or R₃₂₂ and R₇₂₂ can join together to form a ring;

v is 0 to 4; and

each R₂₂ present is independently selected from the group consisting of C₁₋₁₀ alkyl, C₁₋₁₀ alkoxy, hydroxy, halogen, and trifluoromethyl;

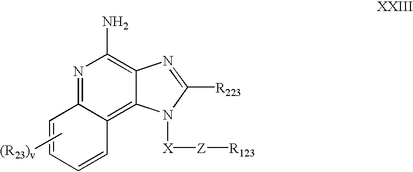

wherein:

X is —CHR₃₂₃—, —CHR₃₂₃-alkyl-, or —CHR₃₂₃-alkenyl-;

Z is —S—, —SO—, or —SO₂—;

R₁₂₃ is selected from the group consisting of:

-alkyl;

-aryl;

-heteroaryl;

-heterocyclyl;

-alkenyl;

—R₄₂₃-aryl;

—R₄₂₃-heteroaryl;

—R₄₂₃-heterocyclyl;

R₂₂₃ is selected from the group consisting of:

-hydrogen;

-alkyl;

-alkenyl;

-aryl;

-heteroaryl;

-heterocyclyl;

-alkyl-Y-alkyl;

-alkyl-Y-alkenyl;

-alkyl-Y-aryl; and

-alkyl or alkenyl substituted by one or more substituents selected from the group consisting of:

—OH;

-halogen;

—N(R₃₂₃)₂;

—CO—N(R₃₂₃)₂;

—CO—C₁₋₁₀ alkyl;

—CO—O—C₁₋₁₀ alkyl;

—N₃;

-aryl;

-heteroaryl;

-heterocyclyl;

—CO-aryl; and

—CO-heteroaryl;

each R₃₂₃ is independently H or C₁₋₁₀ alkyl;

each R₄₂₃ is independently alkyl or alkenyl;

each Y is independently —O— or —S(O)₀₋₂—;

v is 0 to 4; and

each R₂₃ present is independently selected from the group consisting of C₁₋₁₀ alkyl, C₁₋₁₀ alkoxy, hydroxy, halogen and trifluoromethyl;

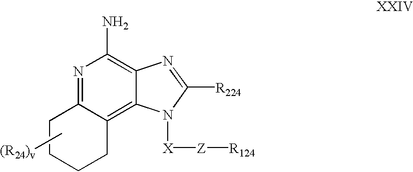

wherein:

X is —CHR₃₂₄—, —CHR₃₂₄-alkyl-, or —CHR₃₂₄-alkenyl-;

Z is —S—, —SO—, or —SO₂—;

R₁₂₄ is selected from the group consisting of:

-alkyl;

-aryl;

-heteroaryl;

-heterocyclyl;

-alkenyl;

—R₄₂₄-aryl;

—R₄₂₄-heteroaryl; and

—R₄₂₄-heterocyclyl;

R₂₂₄ is selected from the group consisting of:

-hydrogen;

-alkyl;

-alkenyl;

-aryl;

-heteroaryl;

-heterocyclyl;

-alkyl-Y-alkyl;

-alkyl-Y-alkenyl;

-alkyl-Y-aryl; and

-alkyl or alkenyl substituted by one or more substituents selected from the group consisting of:

—OH;

-halogen;

—N(R₃₂₄)₂;

—CO—N(R₃₂₄)₂;

—CO—C₁₋₁₀ alkyl;

—CO—O-C₁₋₁₀ alkyl;

—N₃;

-aryl;

-heteroaryl;

-heterocyclyl;

—CO-aryl; and

—CO-heteroaryl;

each R₃₂₄ is independently H or C₁₋₁₀ alkyl;

each R₄₂₄ is independently alkyl or alkenyl;

each Y is independently —O— or —S(O)₀₋₂—;

v is 0 to 4; and

each R₂₄ present is independently selected from the group consisting of C₁₋₁₀ alkyl, C₁₋₁₀ alkoxy, hydroxy, halogen and trifluoromethyl;

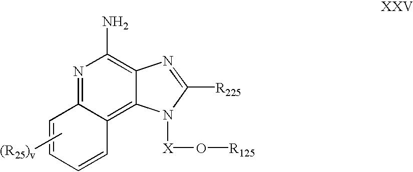

wherein:

X is —CHR₅₂₅—, —CHR₅₂₅-alkyl-, or —CHR₅₂₅-alkenyl-;

R₁₂₅ is selected from the group consisting of:

—R₄₂₅—NR₈₂₅—CR₃₂₅—NR₅₂₅-Z-R₆₂₅-alkyl;

—R₄₂₅—NR₈₂₅—CR₃₂₅—NR₅₂₅-Z-R₆₂₅-alkenyl;

—R₄₂₅—NR₈₂₅—CR₃₂₅—NR₅₂₅Z-R₆₂₅-aryl;

—R₄₂₅—NR₈₂₅—CR₃₂₅—NR₅₂₅-Z-R₆₂₅-heteroaryl;

—R425—NR₈₂₅—CR₃₂₅—NR₅₂₅-Z-R₆₂₅-heterocyclyl;

—R₄₂₅—NR₈₂₅—CR₃₂₅—NR₅₂₅R₇₂₅;

—R₄₂₅—NR₈₂₅—CR₃₂₅—NR₉₂₅-Z-R₆₂₅-alkyl;

—R₄₂₅—NR₈₂₅—CR₃₂₅—NR₉₂₅-Z-R₆₂₅-alkenyl;

—R₄₂₅—NR₈₂₅—CR₃₂₅—NR₉₂₅-Z-R₆₂₅-aryl;

—R₄₂₅—NR₈₂₅—CR₃₂₅—NR₉₂₅-Z-R₆₂₅-heteroaryl; and

—R₄₂₅-NR₈₂₅—CR₃₂₅—NR₉₂₅-Z-R₆₂₅-heterocyclyl;

R₂₂₅ is selected from the group consisting of:

-hydrogen;

-alkyl;

-alkenyl;

-aryl;

-heteroaryl;

-heterocyclyl;

-alkyl-Y-alkyl;

-alkyl-Y-alkenyl;

-alkyl-Y-aryl; and

-alkyl or alkenyl substituted by one or more substituents selected from the group consisting of:

—OH;

-halogen;

—N(R₅₂₅)₂;

—CO—N(R₅₂₅)₂;

—CO—C₁₋₁₀ alkyl;

—CO—O—C₁₋₁₀ alkyl;

—N₃;

-aryl;

-heteroaryl;

-heterocyclyl;

—CO-aryl; and

—CO-heteroaryl;

each R₃₂₅ is ═O or ═S;

each R₄₂₅ is independently alkyl or alkenyl, which may be interrupted by one or more —O— groups;

each R₅₂₅ is independently H or C₁₋₁₀ alkyl;

R₆₂₅ is a bond, alkyl, or alkenyl, which may be interrupted by one or more —O— groups;

R₇₂₅ is H or C₁₋₁₀ alkyl which may be interrupted by a hetero atom, or R₇₂₅ can join with R₅₂₅ to form a ring;

R₈₂₅ is H, C₁₋₁₀ alkyl, or arylalkyl; or R₄₂₅ and R₈₂₅ can join together to form a ring;

R₉₂₅ is C₁₋₁₀ alkyl which can join together with R₈₂₅ to form a ring; each Y is independently —O— or —S(O)₀₋₂—;

Z is a bond, —CO—, or —SO₂—;

v is 0 to 4; and

each R₂₅ present is independently selected from the group consisting of C₁₋₁₀ alkyl, C₁₋₁₀ alkoxy, hydroxy, halogen and trifluoromethyl;

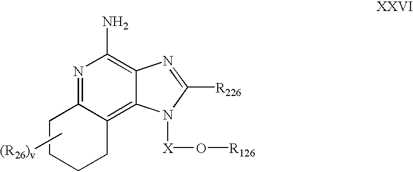

wherein:

X is —CHR₅₂₆—, —CHR₅₂₆-alkyl-, or —CHR₅₂₆-alkenyl-;

R₁₂₆ is selected from the group consisting of:

—R₄₂₆—N₈₂₆—CR₃₂₆—NR₅₂₆-Z-R₆₂₆-alkyl;

—R₄₂₆—NR₈₂₆—CR₃₂₆—NR₅₂₆-Z-R₆₂₆-alkenyl;

—R₄₂₆—NR₈₂₆—CR₃₂₆—NR₅₂₆-Z-R₆₂₆-aryl;

—R₄₂₆—NR₈₂₆—CR₃₂₆—NR₅₂₆-Z-R₆₂₆-heteroaryl;

—R₄₂₆—NR₈₂₆—CR₃₂₆—NR₅₂₆-Z-R₆₂₆-heterocyclyl;

—R₄₂₆—NR₈₂₆—CR₃₂₆—NR₅₂₆R₇₂₆;

—R₄₂₆—NR₈₂₆—CR₃₂₆—NR₉₂₆-Z-R₆₂₆-alkyl;

—R₄₂₆—NR₈₂₆—CR₃₂₆—NR₉₂₆-Z-R₆₂₆-alkenyl;

—R₄₂₆—NR₈₂₆—CR₃₂₆—NR₉₂₆-Z-R₆₂₆-aryl;

—R₄₂₆—NR₈₂₆—CR₃₂₆—NR₉₂₆ Z-R₆₂₆-heteroaryl; and

—R₄₂₆—NR₈₂₆—CR₃₂₆—NR₉₂₆-Z-R626-heterocyclyl;

R₂₂₆ is selected from the group consisting of:

-hydrogen;

-alkyl;

-alkenyl;

-aryl;

-heteroaryl;

-heterocyclyl;

-alkyl-Y-alkyl;

-alkyl-Y-alkenyl;

-alkyl-Y-aryl; and

-alkyl or alkenyl substituted by one or more substituents selected from the group consisting of:

—OH;

-halogen;

—N(R₅₂₆)₂;

—CO—N(R₅₂₆)₂;

—CO—C₁₋₁₀ alkyl;

—CO—O—C₁₋₁₀ alkyl;

—N₃;

-aryl;

-heteroaryl;

-heterocyclyl;

—CO-aryl; and

—CO-heteroaryl;

each R₃₂₆ is ═O or ═S;

each R₄₂₆ is independently alkyl or alkenyl, which may be interrupted by one or more —O— groups;

each R₅₂₆ is independently H or C₁₋₁₀ alkyl;

R₆₂₆ is a bond, alkyl, or alkenyl, which may be interrupted by one or more —O— groups;

R₇₂₆ is H or C₁₋₁₀ alkyl which may be interrupted by a hetero atom, or R₇₂₆ can join with R₅₂₆ to form a ring;

R₈₂₆ is H, C₁₋₁₀ alkyl, or arylalkyl; or R₄₂₆ and R₈₂₆ can join together to form a ring;

R₉₂₆ is C₁₋₁₀ alkyl which can join together with R₈₂₆ to form a ring;

each Y is independently —O— or —S(O)₀₋₂—;

Z is a bond, —CO—, or —SO₂—;

v is 0 to 4; and

each R₂₆ present is independently selected from the group consisting of C₁₋₁₀ alkyl, C₁₋₁₀ alkoxy, hydroxy, halogen, and trifluoromethyl;

and pharmaceutically acceptable salts of any of the foregoing.

Additional suitable 1H-imidazo[4,5-c]pyridin-4-amines include compounds defined by Formula XXVII

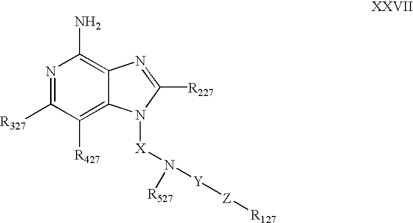

wherein

X is alkylene or alkenylene;

Y is —CO—, —CS—, or —SO₂—;

Z is a bond, —O—, —S—, or —NR₅₂₇—;

R₁₂₇ is aryl, heteroaryl, heterocyclyl, C₁₋₂₀ alkyl or

C₂₋₂₀ alkenyl, each of which may be unsubstituted or substituted by one or more substituents independently selected from the group consisting of:

-alkyl;

-alkenyl;

-aryl;

-heteroaryl;

-heterocyclyl;

-substituted cycloalkyl;

—O-alkyl;

—O-(alkyl)₀₋₁-aryl;

—O-(alkyl)₀₋₁-heteroaryl;

—O-(alkyl)₀₋₁-heterocyclyl;

—COOH;

—CO—O-alkyl;

—CO-alkyl;

—S(O)₀₋₂-alkyl;

—S(O)₀₋₂-(alkyl)₀₋₁-aryl;

—S(O)₀₋₂-(alkyl)₀₋₁-heteroaryl;

—S(O)₀₋₂-(alkyl)₀₋₁-heterocyclyl;

-(alkyl)₀₋₁-N(R₅₂₇)₂;

-(alkyl)₀₋₁-NR₅₂₇—CO—O-alkyl;

-(alkyl)₀₋₁-NR₅₂₇—CO-alkyl;

-(alkyl)₀₋₁-NR₅₂₇—CO-aryl;

-(alkyl)₀₋₁-NR₅₂₇—CO-heteroaryl;

—N₃;

-halogen;

-haloalkyl;

-haloalkoxy;

—CO-haloalkyl;

—CO-haloalkoxy;

—NO₂;

—CN;

—OH;

—SH; and in the case of alkyl, alkenyl, and heterocyclyl, oxo;

R₂₂₇ is selected from the group consisting of:

-hydrogen;

-alkyl;

-alkenyl;

-alkyl-O-alkyl;

-alkyl-S-alkyl;

-alkyl-O-aryl;

-alkyl-S-aryl:

-alkyl-O-alkenyl;

-alkyl-S-alkenyl; and

-alkyl or alkenyl substituted by one or more substituents selected from the group consisting of:

—OH;

-halogen;

—N(R₅₂₇)₂;

—CO—N(R₅₂₇)₂;

—CS—N(R₅₂₇)₂;

—SO₂—N(R₅₂₇)₂;

—NR₅₂₇-CO—C₁₋₁₀ alkyl;

—NR₅₂₇—CS—C₁₋₁₀ alkyl;

—NR₅₂₇—SO₂—C₁₋₁₀ alkyl;

—CO—C₁₋₁₀ alkyl;

—CO—O—C₁₋₁₀ alkyl;

—N₃;

-aryl;

-heteroaryl;

-heterocyclyl;

—CO-aryl; and

—CO-heteroaryl;

R₃₂₇ and R₄₂₇ are independently selected from the group consisting of hydrogen, alkyl, alkenyl, halogen, alkoxy, amino, alkylamino, dialkylamino and alkylthio;

each R₅₂₇ is independently H or C₁₋₁₀alkyl; and pharmaceutically acceptable salts thereof.

As used herein, the terms “alkyl”, “alkenyl” and the prefix “alk-” are inclusive of both straight chain and branched chain groups and of cyclic groups, i.e. cycloalkyl and cycloalkenyl. Unless otherwise specified, these groups contain from 1 to 20 carbon atoms, with alkenyl groups containing from 2 to 20 carbon atoms. Preferred groups have a total of up to 10 carbon atoms. Cyclic groups can be monocyclic or polycyclic and preferably have from 3 to 10 ring carbon atoms. Exemplary cyclic groups include cyclopropyl, cyclopropylmethyl, cyclopentyl, cyclohexyl and adamantyl.

The term “haloalkyl” is inclusive of groups that are substituted by one or more halogen atoms, including perfluorinated groups. This is also true of groups that include the prefix “halo-”. Examples of suitable haloalkyl groups are chloromethyl, trifluoromethyl, and the like.

The term “aryl” as used herein includes carbocyclic aromatic rings or ring systems. Examples of aryl groups include phenyl, naphthyl, biphenyl, fluorenyl and indenyl. The term “heteroaryl” includes aromatic rings or ring systems that contain at least one ring hetero atom (e.g., O, S, N). Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, and so on.

“Heterocyclyl” includes non-aromatic rings or ring systems that contain at least one ring hetero atom (e.g., O, S, N) and includes all of the fully saturated and partially unsaturated derivatives of the above mentioned heteroaryl groups. Exemplary heterocyclic groups include pyrrolidinyl, tetrahydrofuranyl, morpholinyl, thiomorpholinyl, piperidinyl, piperazinyl, thiazolidinyl, imidazolidinyl, isothiazolidinyl, and the like.

Maturation of pDCs

The IRM compounds described above have been found to induce the maturation of plasmacytoid dendritic cells ex vivo. In general, mature pDCs display properties such as cytokine secretion, the expression of particular cell surface markers, and an enhanced ability to stimulate T-cells.

Plasmacytoid dendritic cells that can be matured using the method of the invention can be obtained from any suitable source. For example, the immature pDCs can be obtained by isolating pDCs from tissues such as blood or lymphoid tissues. One method of obtaining pDCs includes isolation of peripheral blood mononuclear cells (PBMCs) from blood and then selectively enriching the sample for pDCs. As used herein, “enrich,” “enriching,” or “enriched” refers to any selective increase in the percentage of one cell type in a population over the percentage of the same cell type in a native sample. A cell population may be enriched by removing other cell types from a cell population. Alternatively, a desired cell type may be selectively removed from a cell population, undesired cells washed away, and the desired cells resuspended in an appropriate cell culture medium. The term “enriched” does not imply that a desired cell type makes up any particular percentage of the relevant cell population.

The pDCs thus obtained will be in an immature state, generally possessing a high capability for antigen capture and processing, but relatively low T-cell stimulatory capacity. To acquire optimal T-cell stimulating capacity, the pDC must be in a stable, mature state. Mature pDCs can be identified by a number of properties, including their expression of certain cell surface markers such as CD40, CD80, CD86 and CCR7. Mature pDCs also exhibit typical behaviors during a mixed lymphocyte reaction including but not limited to increased production of dendritic cell cytokines and induction of cytokine production by T-cells.

The methods of the invention generally include the maturation of pDCs in an isolated cell population by stimulating the pDCs with an IRM in an amount and for a time sufficient to cause the DC to mature. As used herein, “isolated” cell population refers to cells cultured ex vivo. The pDCs may be obtained from a subject by any suitable method including, for example, from a blood sample. The blood sample may be treated in some manner to enrich the percentage of pDCs in the isolated cell population, but such treatment is not required. Thus, “isolated” refers to isolation form the subject and does not relate to any standard of purity of pDCs with respect to any other cell types that may be present in the cell population. Tissue culture medium and conditions are readily determinable to those of skill in the art.

The specific amount of IRM used and the time of exposure will vary according to a number of factors that will be appreciated by those of skill in the art, including the origin of the pDCs to be matured, the potency and other characteristics of the IRM compound used, and so on. In some embodiments, the IRM may be used at a concentration of about 0.1 μM to about 100 μM. The IRM compound may be solubilized before being added to the pDC culture, preferably in water or a physiological buffer. However, if necessary the compound can be solubilized in a small amount of an organic solvent such as DMSO and then diluted or added directly to the pDC culture.

Use of IRM Matured Dendritic Cells

Dendritic cells that have been matured by exposure to certain IRMs have enhanced antigen presenting ability as compared to immature pDCs and can be used in a variety of ways to enhance the immune response of a subject. For example, the mature pDCs can be injected directly into a patient. In this case, it may be desirable that the patient be the source of the pDCs.

The pDCs also can be used in a number of immunotherapies. Examples of such therapies include ex vivo cell transplantation therapies for treating disorders of the immune system, such as AIDS; the ex vivo expansion of T-cells, particularly antigen specific T-cells which can then be used to treat disorders characterized by deterioration of the immune system; the generation of monoclonal antibodies that recognize pDC-specific markers; the preparation of antigen-activated pDCs according to methods known in the art; and development of vaccines and vaccine adjuvants.

Preferred uses of pDCs that have been matured by exposure to one or more IRMs include those that make use of antigen-activated pDC and/or pDC-modified antigens. The antigen-activated pDC, or cellular adjuvants, of the invention are generally prepared by exposing pDC treated with an IRM to an antigen. The antigen may be protein, carbohydrate or nucleic acid in nature and may be derived from any suitable source, including but not limited to neoplastic cells (e.g., tumor cells), prions, and infectious agents (e.g., bacterium, virus, yeast, parasite). Alternatively, the antigen can be derived by recombinant means.

The cellular adjuvant of the invention can be used in the treatment of diseases. For example, cellular adjuvants prepared by exposing pDCs to tumor-derived antigens can be administered to a patient, thereby provoking an anti-tumor immune response in the patient. Similarly, infectious diseases can be treated by administering to the patient cellular adjuvants prepared by exposing the pDC to antigens derived from the infectious agent. The cellular adjuvants also may be used for treatment of non-infectious protein-related diseases including but not limited to Alzheimer's disease and certain forms of heart disease.

Plasmacytoid dendritic cells that have been treated by the method of the invention produce cytokines such as IFN-α that favor the generation of Th1 immune responses. The ability to bias the immune response towards Th1 immunity, as opposed to Th2 immunity, can provide a means for treatment of Th2 mediated diseases. Examples of such diseases include asthma; allergic rhinitis; systemic lupus erythematosis; eczema; atopic dermatitis Ommen's syndrome (hyperseosinophilia syndrome); certain parasitic infections such as cutaneous and systemic leishmaniais, toxoplasma infection and trypanosome infection; certain fungal infections, for example candidiasis and histoplasmosis; and certain intracellular bacterial infections such as leprosy and tuberculosis.

In addition, the ability to induce IL-10 from T-cells can bias the immune response towards a Th3-like response. Th3-like immunity results from the generation of IL-10 producing cells that down-regulate immune responses. These T-cells have also been referred to as regulatory T-cells. The activation of pDC under some circumstances has resulted in the generation of regulatory T-cells which down-regulate effector T-cell function. The generation of such cells may be useful for treatment of disorders mediated solely, or at least in part, by T-cells. Examples of these diseases include, but are not limited to, psoriasis, inflammatory bowl disease, rheumatoid arthritis, diabetes, multiple sclerosis and other diseases associated with chronic T-cell activation.

Generally, the present invention involves treating a cell population of isolated plasmacytoid dendritic cells with an immune response modifier molecule that is an agonist of TLR-6, TLR-7 or TLR-8. Certain embodiments utilize an immune response modifier molecule that is an agonist of TLR-7. Treatment of isolated pDCs in this way induces a broad spectrum of biological activity. The present invention involves methods of treating pDCs to exhibit desired biological activities, methods of detecting desired biological activities, methods of screening cells possessing desired biological activities, cell populations enriched for cells possessing desired biological activities and methods of using enriched cell populations for therapeutic or prophylactic purposes.

In one embodiment, the present invention involves a method of inducing antigen presentation, ex vivo, of a particular antigen by plasmacytoid dendritic cells. The method includes exposing an isolated cell population to an antigen and treating the isolated cell population with an IRM. The IRM treatment enhances the ability of the pDCs to stimulate T-cells. One target for antigen presentation by pDCs is naive T-cells. Thus, one may detect the induction of antigen presentation in pDCs by IRM treatment by detecting one or more biological activities of T-cells that result from contact with a pDC that is presenting antigen. Suitable T-cell biological activities include but are not limited to production of IFN-γ and IL-10.

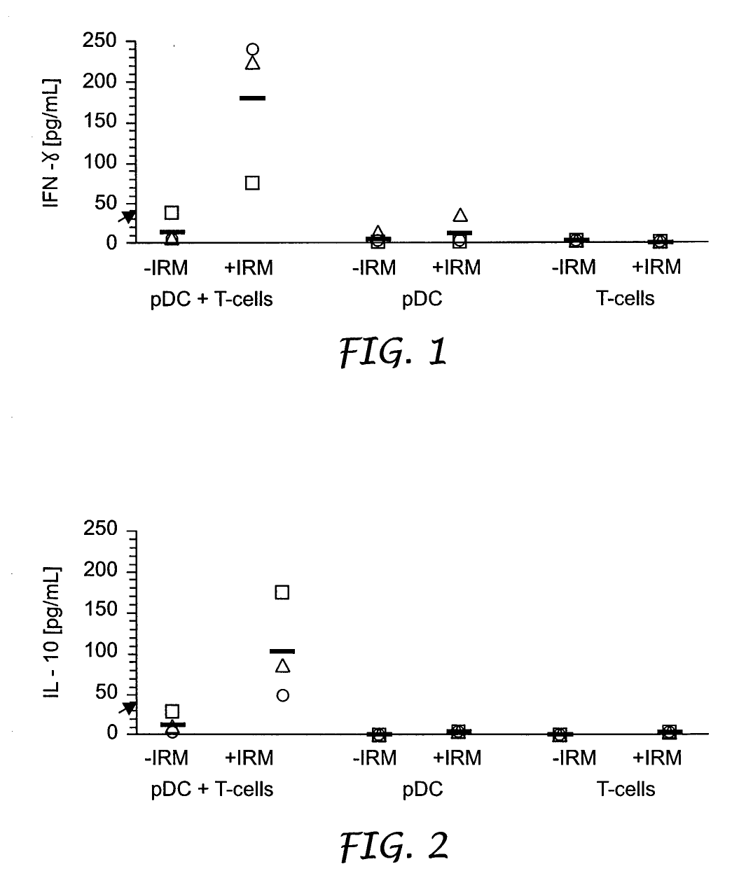

Thus, one method of detecting the induction of antigen presentation by pDCs includes detecting the production of IFN-γ, IL-10, or both by T-cells that have been contacted with pDCs that have been exposed to a particular antigen and treated with an IRM. T-cell production of IFN-γ can be associated with a Th1, or cell-mediated, immune response. IL-10 is one example of a cytokine produced by T-cells in association with a Th2, or humoral, immune response. T-cell production of IL-10 is also associated with a Th3, or regulatory, T-cell response. FIG. 1 shows the results of ELISA detection of IFN-γ production by T-cells in four subjects as a result of contact with pDCs treated with IRM. FIG. 2 shows the results of ELISA detection of IL-10 production by T-cells in four subjects as a result of contact with pDCs treated with IRM.

Isolated pDCs may be treated with any of the IRMs described above. Further, the antigen to which the pDCs are exposed may be any antigen against which a Th1 or Th2 immune response may be desired. Examples of suitable antigens include antigens derived from pathogens, antigens derived from neoplastic cells, and recombinant antigens, as well as other disease-related antigens. Thus, pDC presentation of pathogen antigens may provide therapy or prophylaxis against pathogenic diseases. Similarly, pDC presentation of antigens derived from neoplastic cells may provide therapy or prophylaxis against tumor-related diseases.

Treatment of a subject may include ex vivo antigen presentation by mature pDCs to naive T-cells, followed by administration into the subject of the activated T-cells, the antigen presenting pDCs, or both.

In another embodiment, the present invention provides a method of obtaining a population of mature plasmacytoid dendritic cells by in vivo treatment with an IRM followed by isolation of the matured pDCs from the subject. In certain embodiments, the matured pDCs are isolated from a blood sample taken from the subject. Mature pDCs obtained in this way may be useful for stimulating T-cells ex vivo against one or more antigens to which pDCs have been exposed in vivo, thereby providing the possibility of a subject-specific, antigen-specific therapy.

In another embodiment, the present invention provides a method of detecting cytokine production by isolated plasmacytoid dendritic cells in response to treatment with an IRM. The method includes treating an isolated population of pDCs with an IRM and detecting the production of one or more cytokines. Cytokines produced by pDCs in response to treatment with IRMs include but are not limited to IL-8, IP-10, IL-6, MIP-1α and IFN-ω. Cytokine production may be detected by any one of several standard methods including but not limited to flow cytometry, ELISA, Western blot analysis, and detection of intracellular mRNA that encodes for a particular cytokine.

In another embodiment, the present invention provides a method for detecting expression of co-stimulatory markers by pDCs in response to treatment with an IRM. The method includes treating an isolated population of pDCs with an IRM and detecting the expression of one or more co-stimulatory markers. Examples of co-stimulatory markers that may be detected following pDC treatment with an IRM include but are not limited to CD80, CD86 and CD40. Co-stimulatory marker expression may be detected, for example, by flow cytometry, immunohistochemistry, or detecting intracellular mRNA that encodes a particular co-stimulatory marker.

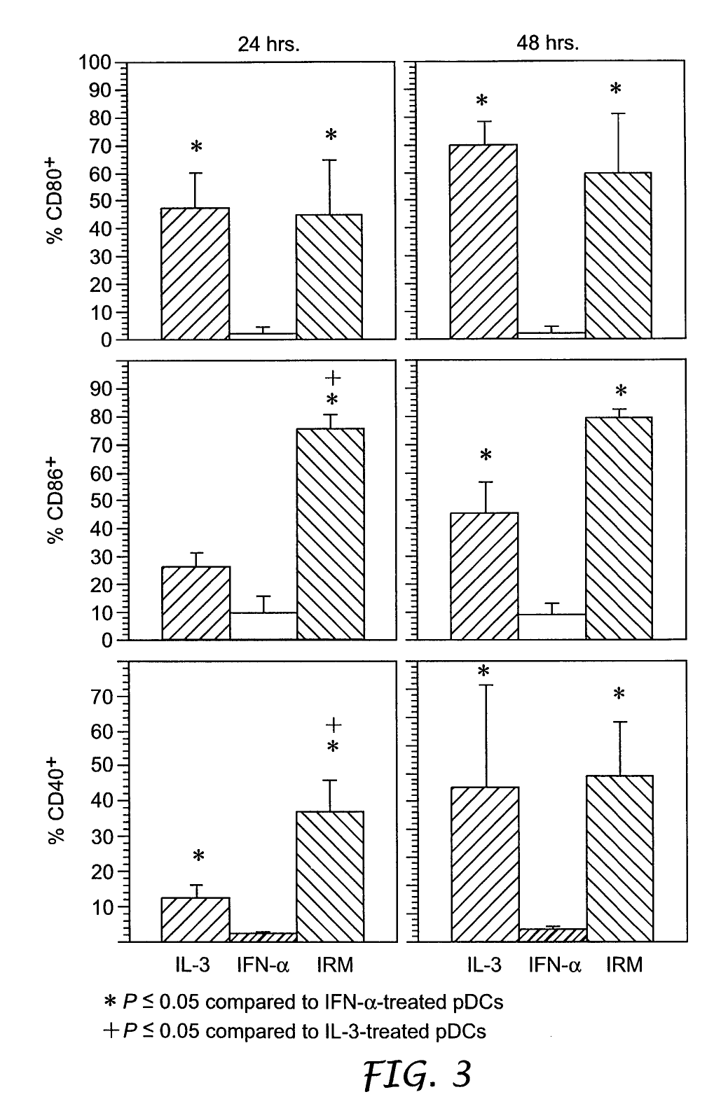

FIG. 3 shows flow cytometry analysis of co-stimulatory marker expression of pDCs treated with IRM compared to pDC expression of co-stimulatory markers when treated with cytokines IL-3 and IFN-α each of which induces pDC survival.

Co-stimulatory markers are expressed on antigen-presenting cells including pDCs to aid antigen presentation to naive T-cells as well as activated and memory T-cells. Thus, detection of expression of co-stimulatory markers may be desirable for detecting pDCs capable of antigen presentation. Also, expression of CCR7 correlates with pDC production of type I interferons and pDC maturation. In yet another embodiment, the present invention provides a method of enhancing survival of pDCs in vitro. The method includes treating a population of isolated pDCs with an IRM and incubating the cells under conditions that promote pDC survival.

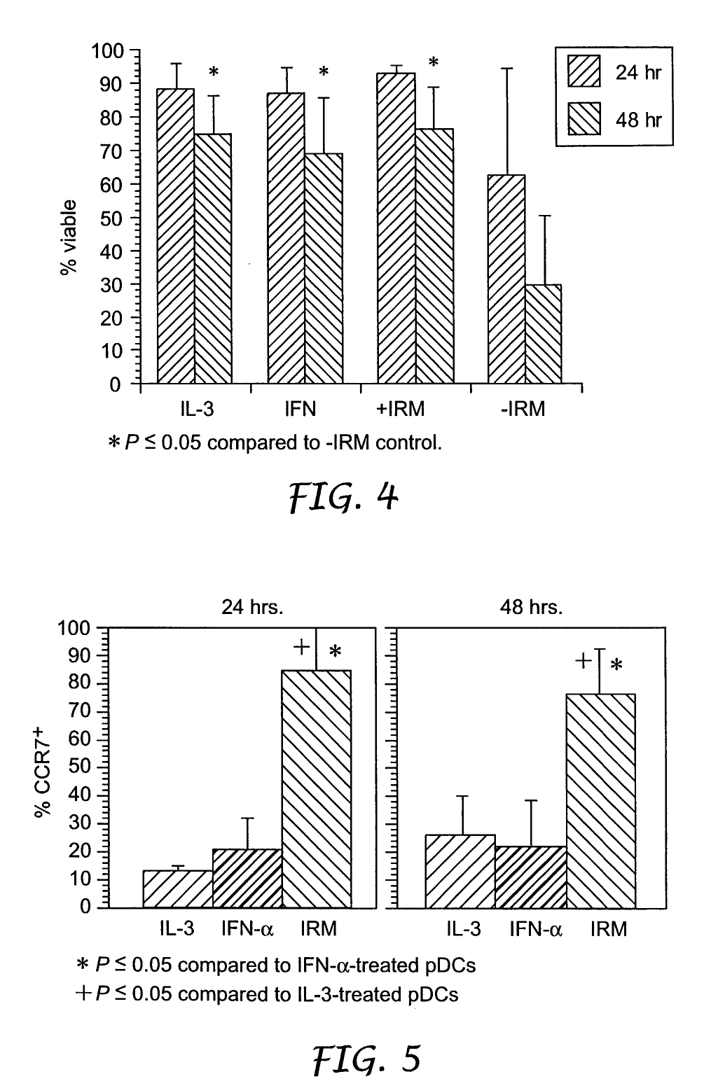

FIG. 4 compares pDC survival at 24 hours and 48 hours after treatment with and without IRM. At 48 hours, pDCs treated with IRM exhibited a statistically significant higher rate of survival. In certain embodiments, pDC survival after 48 hours when treated with IRM is greater than about 75%; in other embodiments, 48-hour survival is greater than about 70%; in other embodiments, 48-hour survival after IRM treatment is greater than about 50%; and in other embodiments, 48-hour survival is greater than about 30%.

Enhanced survival of pDCs in vitro may be desirable when generating a pDC cell population for therapeutic or prophylactic use. Enhanced in vitro survival of pDCs in such cell populations may provide more effective therapy or prophylaxis and may reduce waste associated with expired cell populations.

In yet another embodiment, the present invention provides a method of detecting expression of chemokine receptors by pDCs in response to treatment with an IRM. The method includes treating a population of isolated pDCs with an IRM and then detecting the expression of at least one chemokine receptor. Methods of detecting expression of chemokine receptors include those methods described above useful for detecting expression of co-stimulatory markers and cytokines. One example of a chemokine receptor that is expressed in response to treatment of pDCs with an IRM is CCR7, which is involved with homing mature pDCs to lymph nodes. FIG. 5 shows flow cytometry analysis of pDC expression of the chemokine receptor CCR7 when treated with IRM versus recombinant versions of pDC survival factors IL-3 and IFN-α.

The present invention also provides a method of preparing a population of pDCs that express a relatively high level of chemokine receptor. This method includes inducing chemokine receptor expression by treating a population of isolated pDCs with an IRM. The method also includes enriching the cell population for cells that express chemokine receptors.

Cells expressing chemokine receptors may migrate, in vivo, to secondary lymphoid tissue, where antigen presentation to T-cells can occur, thereby stimulating Th1 and Th2 immune responses. Antigen-specific pDCs expressing chemokine receptors may provide particularly useful therapeutic or prophylactic agents, either alone or as an adjuvant in a vaccine, for example. Thus the present invention provides a method of treating a disease that includes exposing a population of isolated pDCs to an antigen, treating the pDCs with an IRM, enriching the treated cells for cells that express a chemokine receptor, and administering the enriched cell population to a patient.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

IRM, 4-amino-2-ethoxymethyl-α,α-dimethyl-1H-imidazo[4,5-c]quinoline-1-ethanol, M.W.=314.4, was dissolved in dimethyl sulfoxide (DMSO, sterile cell culture grade, Sigma Chemical Company, St. Louis, Mo.) to form a 12 mM solution of that IRM. The IRM solutions were stored in aliquots at −20° C. Unless otherwise specified, IRM was added to cell cultures to a final concentration of 3 μM.

Unless otherwise indicated, all pDC cell cultures were maintained in X-Vivo 20 medium (BioWhittaker, Inc., Walkersville, Md.) at 37° C. with 5% CO₂.

Antibodies used for positive selection and depletion of pDC include BDCA-2 and BDCA-4 microbeads (Miltenyi Biotec, Inc., Auburn, Calif.). Biotin-labeled monoclonal antibodies were used to obtain pDC by negative selection; these include CD3, CD11b, CD11c, CD14, CD19, CD56 (Ancell Corp., Bayport, Minn.). Antibodies and fluorochrome-labeled reagents for flow cytometry include HLA-DR-PerCP, CD123 (IL-3-Rα)-PE, CD80-PE, CD86-PE, CD40-PE, biotin-labeled CCR7, streptavidin-PE, TNF-α-FITC, TNF-α-PE, IL-12p40/70-FITC, IL-12p40/70-PE (BD Pharmingen, San Diego, Calif.), IFN-α2-FITC and IFN-α2-PE (Chromaprobe Inc., Aptos, Calif.). Non-specific binding to Fe receptors was prevented using IgG (Whole molecule, Pierce Chemical Company, Rockford, Ill.) or FcR blocking reagent (Miltenyi Biotec, Inc.).

Intracellular flow cytometry was performed using the CytoStain Kit containing GolgiPlug (BD Pharmingen).

HSV-1 (MacIntyre) was obtained from American Type Culture Collection (ATCC, Manassas, Va.). LPS was obtained from Sigma Chemical Company, St. Louis, Mo. Recombinant human cytokines IL-3 and rGM-CSF were obtained from R&D Systems, Inc., Minneapolis, Minn. and rIFN-αF was obtained from PBL Biomedical Laboratories, New Brunswick, N.J.

### Example 1

### PBMC Isolation

PMBCs were isolated from whole blood anti-coagulated with EDTA by density gradient centrifugation using Histopaque 1077 (Sigma Chemical Company, St. Louis, Mo.) as recommended by the manufacturer. The isolated mononuclear cells were washed twice with Hank's Balanced Salts Solution (Celox Laboratories, Inc., St. Paul, Minn.) and resuspended in complete RPMI (cRPMI; RPMI 1640, 25 mM HEPES, 1 MM sodium pyruvate, 0.1 mM non-essential amino acids, 1 mM L-glutamine, 1% penicillin/streptomycin, 5×10⁻⁵ M 2-mercaptoethanol and 10% heat-inactivated fetal calf serum (FCS, Celox Laboratories, Inc. or Hyclone Laboratories, Inc., Logan, Utah)) or X-Vivo 20 medium (BioWhittaker, Inc., Walkersville, Md.).

### Example 2

### Plasmacytoid DC Isolation

Human pDCs were isolated from PBMC by immunomagnetic bead positive selection according to the manufacturer's instructions (Miltenyi Biotec, Inc., Auburn, Calif.). Briefly, PBMC were incubated with pDC-specific antibodies, BDCA-2 or BDCA-4, and the labeled cells were collected with a Miltenyi LS column. The positively selected cells were resuspended in X-Vivo 20 medium.

Human pDC were also enriched by negative selection from PBMC by depleting Lin⁺ cells. Briefly, PBMC isolated from 120 mL whole blood were resuspended in 1 mL PBS, 1% BSA, 1 mM EDTA and incubated with biotin-labeled antibodies specific for CD3, CD14, CD19, CD56 and in some cases CD11b and CD11c, at a final concentration of 100 μg/mL for each antibody. After 15 minutes of incubation at 6-12° C., the cells were washed and incubated with either streptavidin microbeads or anti-biotin microbeads for an additional 15 minutes at 6-12° C. After washing, the unlabeled fraction was collected on Miltenyi CS or LS columns and the cells were resuspended in X-Vivo 20. The pDC population, HLA-DR⁺/CD123^{HI}, was routinely 5-10% of the final preparation as compared to 0.1-0.5% of the starting PBMC population.

### Example 3

### Intracellular Cytokine Detection Determined by Flow Cytometry

Cells were incubated at 1×10⁶/mL in X-Vivo 20 medium (BioWhittaker, Inc.) and stimulated with IRM for 1 hour. After stimulation, 1 μL Brefeldin-A (GolgiPlug, BD Pharmingen, San Diego, Calif.) was added for every mL of cell culture medium. The cells were then incubated overnight at 37° C. with 5% CO₂, not exceeding 12 hours. The cells were washed and resuspended in Pharmingen Stain Buffer-BSA (BD Pharmingen) two times. Fc receptors were blocked with ImmunoPure mouse IgG (Whole Molecule, Pierce Chemical Company) (100 mL/10⁶ cells in 100 μL of staining buffer for 15 minutes at 4° C.). Cells were then washed with staining buffer and then stained for surface antigens (10 μL antibody in 50 μL staining buffer for 30 minutes at 4° C.). Cells were then washed and resuspended in Cytofix/Cytoperm (BD Pharmingen) to fix and permeabilize the cells. After washing with Perm/Wash solution (BD Pharmingen), the cells were stained for intracellular cytokines with anti-TNF-α or anti-IFN-α fluorochrome-labeled antibodies for 30-45 minutes at 4° C. Finally, the cells were washed and resuspended in staining Buffer and analyzed using a FACScan FLOW cytometer and CellQuest software (BD Biosciences, San Jose, Calif.).

### Example 4

### Co-Stimulatory Marker Expression Determined by Flow Cytometry

BDCA-2 or BDCA-4 purified cells were treated 24 or 48 hours in X-Vivo 20 medium with 1000 U/mL rIL-3, 1000 U/mL rIFN-α or IRM.

Prior to staining, the cells were washed in Pharmingen Stain Buffer-BSA. The cells were then resuspended in Pharmingen Stain Buffer-BSA and fluorochrome-labeled antibodies specific to CD80, CD86, or CD40 were added. After 30 minutes at 4° C., the cells were washed and analyzed by flow cytometry.

### Example 5

### Chemokine Receptor Expression Determined by Flow Cytometry

BDCA-2 or BDCA-4 cells were purified and treated as described in Example 4, except that the fluorochrome-labeled antibodies were specific to CCR7.

### Example 6

### Cytokine and Chemokine Analysis by Real-Time (RT) PCR and ELISA

Cytokine and chemokine expression were evaluated by RT PCR. PBMC and BDCA-2-purified pDC were stimulated in 24-well plates with 3 μM IRM. Vehicle control cells were treated with DMSO. Cells were incubated for either one or two hours at 37° C. At the indicated times the cells were harvested by gently pipeting the cells into a 1.5 mL Eppendorf tube and centrifuging at 400×g for 10 min at 4° C. The supernatant was removed from the tube and the cells were lysed with 1 mL of TRIzol (Invitrogen Corp., Carlsbad, Calif.). RNA was purified from the samples and treated with DNase I (Invitrogen Corp.) to remove contaminating genomic DNA, after which the samples were re-extracted with TRIzol. Final pellets were suspended in 10 μL of water. 1 μL was diluted 1:100, and the RNA was quantified by absorbency (Abs₂₆₀).

The RNA was reverse-transcribed using SuperScript First Strand Synthesis System for RT-PCR (Invitrogen Corp.). Primers for quantitative PCR were generated using Primer Express (Applied Biosystems Group, Foster City, Calif.). Each primer set was designed to amplify genomic DNA and was tested against a sample of human genomic DNA to verify the amplicon size. The primer sets are shown in Table I. Quantitative PCR was performed on an ABI PRISM™ 7700 Sequence Detector (Applied Biosystems Group). Amplified products were detected using SYBR® Green PCR Master Mix (Applied Biosystems Group). Each primer set was tested in triplicate for each sample. PCR was performed for thirty-five cycles for 15 seconds at 95° C. and 1 minute at 60° C., preceded by incubation for 2 minutes at 50° C. and 10 minutes at 95° C.

The instrument software calculated the number of cycles, designated Cₜ, required for the accumulated signal to reach a designated threshold value at least 10 standard deviations greater than the baseline. The Cₜ value is then proportional to the number of starting copies of the target sequence. Relative quantitation of gene expression was performed using the ΔΔCₜ method (User Bulletin #2, Applied Biosystems Group). Briefly, the fold change in expression was calculated relative the expression of GAPDH using the following formula:

Fold Change=2^{-(ΔΔC}ₜ)

where ΔΔCₜ=[Cₜ gene of interest (stimulated sample)−Cₜ GAPDH (stimulated sample)]−[Cₜ gene of interest (vehicle control)−Cₜ GAPDH (vehicle control)].

Cytokine and chemokine protein levels were measured from tissue culture supernatants or cell extracts by ELISA. Human TNF, IL-12, IL-10 (standard IL-10 assay and IL-10 Ultrasensitive), IL-6, IL-IRA, MCP-1, and Mip-1α ELISA kits were obtained from BioSource International, Inc. (Camarillo, Calif.). Human Mip-3α and Multi-Species IFN-α ELISA kits were obtained from R&D Systems (Minneapolis, Minn.) and PBL Biomedical Laboratories (New Brunswick, N.J.), respectively. Human IP-10 ELISA kits were obtained from Cell Sciences, Inc. (Norwood, Mass.). All ELISA results are expressed in pg/mL. The limit of reliable detection for all ELISA assays is less than or equal to 40 pg/mL, except for IL-10 Ultrasensitive assay which is 1 pg/mL. The Multi-Species IFN-α ELISA assay specifically detects all of the human IFN-A subtypes, except IFN-αF (IFN-α21).

### Example 8

### T-Cell Activation Assay

Frozen naive cord blood CD4⁺/CD45RA⁺/CD45RO⁻ T-cells were obtained from AllCells LLC (Berkeley, Calif.) and thawed according to the manufacturer's recommendation. Briefly, frozen cells were thawed in a 37° C. water bath and transferred to 15 mL conical tubes containing 300 μg DNase I (Stemcell Technologies, Inc., Vancouver, British Columbia). X-Vivo 20 media (BioWhittaker, Inc., Walkersville, Md.) was slowly added to the cells bringing the volume up to 15 mL. The cells were washed two times by centrifugation at 200×g for 15 minutes in X-Vivo 20 medium. Cells were finally resuspended in X-Vivo 20 medium at 2×10⁶ cells/mL.

Plasmacytoid dendritic cells were prepared by positive selection with BDCA-4 microbeads (Miltenyi Biotec, Inc., Auburn, Calif.). The pDC were co-cultured with naive cord blood T-cells at an enriched-pDC to T-cell ratio of 1:10 (1×10⁵ pDC/mL:1×10⁶ T-cells/mL per well) in X-Vivo 20 medium. At the initiation of culture, the cells were treated with IL-3 [1000 U/mL], IFN-α [1000 U/mL], IRM or vehicle (DMSO). After 72 hr, cell-free supernatants were collected and analyzed for IFN-γ, IL-13 and IL-10 by ELISA.

### Example 9

### Enhanced Survival

Isolated pDCs were obtained as described in Example 2. The isolated pDCs were incubated in with and without IRM. Cell viability was measured in both cultures by flow cytometry after 24 hours and again after 48 hours.

### Example 10

### Chemokine Receptor Expression Screening

A population of pDCs can be obtained as described in Example 2. The pDC-containing cell population can be incubated at 1×10⁶/mL in X-Vivo 20 medium (BioWhittaker, Inc.) and stimulated with IRM (1 μM-10 μM) for 1 hour. Chemokine expression can be determined according to the method of either Example 5 or Example 6.

### Example 11

### Treatment Using pDC Population Enriched for Cells Expressing Chemokine Receptor

Plasmacytoid dendritic cells can be obtained from a patient as described in Example 2. The isolated pDCs can be co-stimulated with antigen (e.g., tetanus toxoid) and IRM (1 μM-10 μM) from about 1 hour to about 24 hours.

Stimulated pDCs expressing high levels of chemokine receptor can be screened as described in Example 10. Plasmacytoid dendritic cells expressing high levels of chemokine receptors can be sorted by flow cytometry. The pDCs expressing chemokine receptor can be resuspended in X-Vivo 20 medium.

Plasmacytoid dendritic cells expressing the antigen and expressing high levels of chemokine receptor can be reintroduced to the patient intravenously or by subcutaneous immunization.

Statistical Methods

FIG. 3 shows data that were examined separately for each co-stimulatory marker and time point.

FIG. 4 shows an analysis of variance (ANOVA), with percent viable as the response variable and explanatory variables for donor and treatment, performed on the untransformed and arcsin-transformed data separately for 24 and 48 hour time points. Pairwise comparisons of IRM-treated cells to the control group were performed using the Dunnett adjustment to preserve the overall 0.05 level of significance. If there were discrepancies between the 2 methods, the results from the arcsin transformed data were reported.

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

TABLE I
Real-time RT-PCR primer sets

| Gene | Accession No. | Forward Primer | Reverse Primer |
|---|---|---|---|
| IL-6 | M14584 | AAGCAGCAAAGAGGCACTGG | GCATCCATCTTTTTCAGCCATC |
| IL-10 | M57627 | TGAGAACAGCTGCACCCACTT | GCTGAAGGCATCTCGGAGATC |
| IL-12p40 | NM_002187 | ACAACTTGCAGCTGAAGCCA | AGGGTACTCCCAGCTGACCTC |
| IL-IRA | NM_000577 | GGTTGGTTCCTCTGCACAGC | GCCTTCGTCAGGCATATTGGT |
| TNF-α | M10988 | ATCAATCGGCCCGACTATCTC | CACAGGGCAATGATCCCAA |
| IP-10 | NM_001565 | TACGCTGTACCTGCATCAGCA | GACAAAATTGGCTTGCAGGAAT |
| MCP-1 | NM_002982 | AGCAAGTGTCCCAAAGAAGCTG | CAGATCTCCTTGGCCACAATG |
| MIP-1α | NM_002983 | AGCTACACCTCCCGGCAGAT | GGCTGCTCGTCTCAAAGTAGTCA |
| MIP-3α | NM_004591 | GCTGTCTTGGATACACAGACCGT | CACAGCCTTCATTGGCCAG |
| GAPDH | | ACCCACTCCTCCACCTTTGA | TGACAAAGTGGTCGTTGAGGG |

What is claimed is:

1. A method of enhancing antigen presentation by dendritic cells in vitro, the method comprising:
(a) exposing an isolated dendritic cell population to an antigen;
(b) contacting the isolated dendritic cell with an immune response modifier molecule that is an agonist of Toll-like receptor 6, Toll-like receptor 7 or Toll-like receptor 8; and
(c) allowing the dendritic cell to process and present the antigen.

2. The method of claim 1 wherein the antigen is derived from neoplastic cells, derived from an infectious agent, or is recombinantly derived.

3. The method of claim 1 wherein the immune response modifier molecule is an agonist of Toll-like receptor 7.

4. The method of claim 1 wherein the immune response modifier molecule is selected from the group consisting of imidazoquinoline amines, imidazopyridine amines, 6,7-fused cycloalkylimidazopyridine amines, 1,2-bridged imidazoquinoline amines, thiazolo- and oxazolo-quinolinamines and pyridinamines, imidazonaphthyridine amines and tetrahydroimidazonaphthyridine amines, and pharmaceutically acceptable salts thereof.

5. The method of claim 4 wherein the immune response modifier molecule is selected from the group consisting of imidazoquinoline amines and 6,7-fused cycloalkylimidazopyridine amines, and pharmaceutically acceptable salts thereof.

6. The method of claim 1 further comprising detecting the antigen presentation.

7. The method of claim 6 wherein detecting antigen presentation comprises:
(a) contacting the activated dendritic cells with naive T-cells; and
(b) detecting production of one or more cytokines that are produced by T-cells as a result of antigen presentation by dendritic cells.

8. The method of claim 7 wherein the one or more cytokines comprise IFN-γ or IL-10.

9. The method of claim 1 wherein the dendritic cells are plasmacytoid dendritic cells.

10. An isolated dendritic cell population produced by the process of:
(a) exposing an isolated dendritic cell population to an antigen;
(b) contacting the isolated dendritic cell with an immune response modifier molecule that is an agonist of Toll-like receptor 6, Toll-like receptor 7 or Toll-like receptor 8; and
(c) allowing the dendritic cell to process and express the antigen.

11. The method of claim 10 wherein the immune response modifier molecule is an agonist of Toll-like receptor 7.

12. The method of claim 10 wherein the immune response modifier molecule is selected from the group consisting of imidazoquinoline amines, imidazopyridine amines, 6,7-fused cycloalkylimidazopyridine amines, 1,2-bridged imidazoquinoline amines, thiazolo- and oxazolo-quinolinamines and pyridinamines, imidazonaphthyridine amines and tetrahydroimidazonaphthyridine amines, and pharmaceutically acceptable salts thereof.

13. The method of claim 12 wherein the immune response modifier molecule is selected from the group consisting of imidazoquinoline amines and 6,7-fused cycloalkylimidazopyridine amines, and pharmaceutically acceptable salts thereof.

14. The cell population of claim 10 wherein the antigen is derived from neoplastic cells, derived from an infectious agent, or is recombinantly derived.

15. The cell population of claim 10 wherein the dendritic cells are plasmacytoid dendritic cells.

16. A method of obtaining a population of mature dendritic cells, the method comprising:
(a) administering an immune response modifier molecule that is an agonist of Toll-like receptor 6, Toll-like receptor 7 or Toll-like receptor 8 to a subject in an amount effective to mature dendritic cells of the subject; and
(b) isolating the mature dendritic cells.

17. The method of claim 16 wherein the mature dendritic cells are isolated from a blood sample of the subject.

18. The method of claim 16 wherein the amount of immune response modifier molecule administered to the subject is at least 0.001 mg/kg.

19. The method of claim 16 wherein the dendritic cells are plasmacytoid dendritic cells.

20. A cell population obtained by the method of claim 16.

21. The method of claim 16 wherein the immune response modifier molecule is an agonist of Toll-like receptor 7.

22. The method of claim 16 wherein the immune response modifier molecule is selected from the group consisting of imidazoquinoline amines, imidazopyridine amines, 6,7-fused cycloalkylimidazopyridine amines, 1,2-bridged imidazoquinoline amines, thiazolo- and oxazolo-quinolinamines and pyridinamines, imidazonaphthyridine amines and tetrahydroimidazonaphthyridine amines, and pharmaceutically acceptable salts thereof.

23. The method of claim 22 wherein the immune response modifier molecule is selected from the group consisting of imidazoquinoline amines and 6,7-fused cycloalkylimidazopyridine amines, and pharmaceutically acceptable salts thereof.

24. A method of detecting cytokine production by a plasmacytoid dendritic cell, the method comprising:
(a) contacting an isolated plasmacytoid dendritic cell with an immune response modifier molecule that is an agonist of Toll-like receptor 6, Toll-like receptor 7 or Toll-like receptor 8 in an amount effective for inducing the plasmacytoid dendritic cell to produce one or more cytokines selected from IL-8, IP-10, IL-6, MIP-1α, and IFN-ω; and
(b) detecting production of at least one of the cytokines by the dendritic cell.

25. The method of claim 24 wherein the immune response modifier molecule is an agonist of Toll-like receptor 7.

26. The method of claim 24 wherein the immune response modifier molecule is selected from the group consisting of imidazoquinoline amines, imidazopyridine amines, 6,7-fused cycloalkylimidazopyridine amines, 1,2-bridged imidazoquinoline amines, thiazolo- and oxazolo-quinolinamines and pyridinamines, imidazonaphthyridine amines and tetrahydroimidazonaphthyridine amines, and pharmaceutically acceptable salts thereof.

27. The method of claim 26 wherein the immune response modifier molecule is selected from the group consisting of imidazoquinoline amines and 6,7-fused cycloalkylimidazopyridine amines, and pharmaceutically acceptable salts thereof.

28. The method of claim 24 wherein the amount of immune response modifier molecule is provided at a concentration of at least about 0.001 μM.

29. The method of claim 24 wherein the step of detecting production of at least one of the cytokines comprises detecting intracellular cytokine by flow cytometry.

30. The method of claim 24 wherein the step of detecting production of at least one of the cytokines comprises detecting extracellular cytokine.

31. The method of claim 24 wherein the step of detecting production of at least one of the cytokines comprises using an enzyme-linked immunosorbent assay.

32. The method of claim 24 wherein the step of detecting production of at least one of the cytokines comprises detecting mRNA that encodes the cytokine in the plasmacytoid dendritic cell.

33. A method of detecting expression of co-stimulatory markers by plasmacytoid dendritic cells, the method comprising:
(a) contacting an isolated plasmacytoid dendritic cell with an immune response modifier molecule that is an agonist of Toll-like receptor 6, Toll-like receptor 7 or Toll-like receptor 8 in an amount effective for inducing the plasmacytoid dendritic cell to express one or more co-stimulatory marker; and
(b) detecting the expression of at least one co-stimulatory marker by the plasmacytoid dendritic cell.

34. The method of claim 33 wherein the immune response modifier molecule is an agonist of Toll-like receptor 7.

35. The method of claim 33 wherein the immune response modifier molecule is selected from the group consisting of imidazoquinoline amines, imidazopyridine amines, 6,7-fused cycloalkylimidazopyridine amines, 1,2-bridged imidazoquinoline amines, thiazolo- and oxazolo-quinolinamines and pyridinamines, imidazonaphthyridine amines and tetrahydroimidazonaphthyridine amines, and pharmaceutically acceptable salts thereof.

36. The method of claim 35 wherein the immune response modifier molecule is selected from the group consisting of imidazoquinoline amines and 6,7-fused cycloalkylimidazopyridine amines, and pharmaceutically acceptable salts thereof.

37. The method of claim 33 wherein the amount of immune response modifier molecule is provided at a concentration of at least 0.001 μM.

38. The method of claim 33 wherein the co-stimulatory marker comprises CD80, CD86, CD40, or HLA-DR.

39. The method of claim 33 wherein the step of detecting expression of at least one co-stimulatory marker comprises using flow cytometry.

40. The method of claim 33 wherein the step of detecting expression of at least one co-stimulatory marker comprises immunological detection of at least one co-stimulatory marker on the cell surface of a plasmacytoid dendritic cell.

41. The method of claim 33 wherein the step of detecting expression of at least one co-stimulatory marker comprises detecting mRNA that encodes the co-stimulatory marker in the plasmacytoid dendritic cell.

42. A method of enhancing survival of isolated plasmacytoid dendritic cells, the method comprising:
(a) contacting a population of isolated plasmacytoid dendritic cells with an immune response modifier molecule that is an agonist of Toll-like receptor 6, Toll-like receptor 7 or Toll-like receptor 8 in an amount effective for enhancing survival of the plasmacytoid dendritic cells; and
(b) incubating the plasmacytoid dendritic cells under conditions so that at least 30% of the plasmacytoid dendritic cell survive for at least 48 hours.

43. The method of claim 42 wherein at least 50% of the plasmacytoid dendritic cells survive for at least 48 hours.

44. The method of claim 42 wherein at least 70% of the plasmacytoid dendritic cells survive for at least 48 hours.

45. The method of claim 42 wherein at least 75% of the plasmacytoid dendritic cells survive for at least 48 hours.

46. The method of claim 42 wherein the immune response modifier molecule is an agonist of Toll-like receptor 7.

47. The method of claim 42 wherein the immune response modifier molecule is selected from the group consisting of imidazoquinoline amines, imidazopyridine amines, 6,7-fused cycloalkylimidazopyridine amines, 1,2-bridged imidazoquinoline amines, thiazolo- and oxazolo-quinolinamines and pyridinamines, imidazonaphthyridine amines and tetrahydroimidazonaphthyridine amines, and pharmaceutically acceptable salts thereof.

48. The method of claim 47 wherein the immune response modifier molecule is selected from the group consisting of imidazoquinoline amines and 6,7-fused cycloalkylimidazopyridine amines, and pharmaceutically acceptable salts thereof.

49. A method of detecting expression of chemokine receptors by plasmacytoid dendritic cells, the method comprising:
(a) contacting an isolated plasmacytoid dendritic cell with an immune response modifier molecule that is an agonist of Toll-like receptor 6, Toll-like receptor 7 or Toll-like receptor 8 in an amount effective for inducing the plasmacytoid dendritic cell to express one or more chemokine receptors; and
(b) detecting expression of at least one chemokine receptor.

50. The method of claim 49 wherein the immune response modifier molecule is an agonist of Toll-like receptor 7.

51. The method of claim 49 wherein the immune response modifier molecule is selected from the group consisting of imidazoquinoline amines, imidazopyridine amines, 6,7-fused cycloalkylimidazopyridine amines, 1,2-bridged imidazoquinoline amines, thiazolo- and oxazolo-quinolinamines and pyridinamines, imidazonaphthyridine amines and tetrahydroimidazonaphthyridine amines, and pharmaceutically acceptable salts thereof.

52. The method of claim 51 wherein the immune response modifier molecule is selected from the group consisting of imidazoquinoline amines and 6,7-fused cycloalkylimidazopyridine amines, and pharmaceutically acceptable salts thereof.

53. The method of claim 49 wherein the amount of immune response modifier is provided at a concentration of at least 0.001 μM.

54. The method of claim 49 wherein the chemokine receptor is CCR7.

55. The method of claim 49 wherein the step of detecting expression of at least one chemokine receptor comprises detecting up-regulation of chemokine receptor expression or down-regulation of chemokine receptor expression.

56. The method of claim 55 wherein the step of detecting expression of at least one chemokine receptor comprises the use of flow cytometry.

57. The method of claim 55 wherein the step of detecting expression of at least one chemokine receptor comprises using an enzyme-linked immunosorbent assay.

58. The method of claim 55 wherein the step of detecting expression of at least one chemokine receptor comprises detecting mRNA that encodes the chemokine receptor in the plasmacytoid dendritic cells.

59. A method of identifying a compound that selectively induces production of a chemokine receptor by plasmacytoid dendritic cells, the method comprising:
(a) obtaining a population of cells that includes both inflammatory cytokine producing cells and plasmacytoid dendritic cells;
(b) contacting the population of cells with a test compound;
(c) determining the amount of chemokine receptor present in the population of cells contacted with the test compound;
(d) determining the amount of inflammatory cytokine(s) present in the population of cells contacted with the test compound; and
(e) identifying the test compound as a selective inducer of the chemokine receptor if the chemokine receptor is present in the population of cells after contact with the test compound in an amount at least three times greater than the amount of inflammatory cytokine(s) present in the population of cells.

60. The method of claim 59 wherein the amount of chemokine receptor is determined by flow cytometry.

61. The method of claim 59 wherein the amount of inflammatory cytokine(s) is determined from culture supernatants using an enzyme-linked immunosorbent assay or a bioassay.

62. The method of claim 59 wherein the amounts of chemokine receptor and inflammatory cytokine(s) are determined using one or more methods selected from the group consisting of Northern blotting, Western blotting, and real-time PCR.

63. The method of claim 59 wherein the inflammatory cytokine is TNF-α or IL-12.

64. The method of claim 59 wherein the population of cells is contacted with the test compound at a concentration of from about 0.005 μM to about 5 μM.

65. A method of preparing a cell population enriched for cells that express a chemokine receptor, the method comprising:
(a) contacting an isolated plasmacytoid dendritic cell with an immune response modifier molecule that is an agonist of Toll-like receptor 6, Toll-like receptor 7 or Toll-like receptor 8 in an amount effective for inducing the plasmacytoid dendritic cell to express one or more chemokine receptor; and
(b) enriching the cell population for cells that express a chemokine receptor.

66. The method of claim 65 wherein the immune response modifier molecule is an agonist of Toll-like receptor 7.

67. The method of claim 65 wherein the immune response modifier molecule is selected from the group consisting of imidazoquinoline amines, imidazopyridine amines, 6,7-fused cycloalkylimidazopyridine amines, 1,2-bridged imidazoquinoline amines, thiazolo- and oxazolo-quinolinamines and pyridinamines, imidazonaphthyridine amines and tetrahydroimidazonaphthyridine amines, and pharmaceutically acceptable salts thereof.

68. The method of claim 67 wherein the immune response modifier molecule is selected from the group consisting of imidazoquinoline amines and 6,7-fused cycloalkylimidazopyridine amines, and pharmaceutically acceptable salts thereof.

69. The method of claim 65 wherein the step of enriching the cell population comprises selectively removing cells that do not express chemokine receptor from the cell population.

70. The method of claim 65 wherein the step of enriching the cell population comprises:
(a) contacting the cell population with a substrate that selectively bind cells that express a chemokine receptor to a substrate;
(b) allowing the substrate to reversibly bind cells that express a chemokine receptor;
(c) removing unbound cells; and
(d) collecting the bound cells.

71. The method of claim 70 wherein the selective binding comprises adsorption or immunosorption.

72. The method of claim 65 wherein the chemokine receptor is CCR7.

73. A population of plasmacytoid dendritic cells enriched for cells that express chemokine receptors prepared by the method of claim 65.

74. The cell population of claim 73 wherein the chemokine receptor is CCR7.

75. A method of treating a disease comprising:
(a) contacting an isolated plasmacytoid dendritic cell with an immune response modifier molecule that is an agonist of Toll-like receptor 6, Toll-like receptor 7 or Toll-like receptor 8 in an amount effective for inducing the plasmacytoid dendritic cell to express one or more chemokine receptors;
(b) contacting the population of plasmacytoid dendritic cells with an antigen associated with the disease;
(c) enriching the cell population for cells expressing a high level of at least one chemokine receptor; and
(d) administering the enriched cell population to a patient.

76. The method of claim 75 wherein the immune response modifier molecule is an agonist of Toll-like receptor 7.

77. The method of claim 75 wherein the immune response modifier molecule is selected from the group consisting of imidazoquinoline amines, imidazopyridine amines, 6,7-fused cycloalkylimidazopyridine amines, 1,2-bridged imidazoquinoline amines, thiazolo- and oxazolo-quinolinamines and pyridinamines, imidazonaphthyridine amines and tetrahydroimidazonaphthyridine amines, and pharmaceutically acceptable salts thereof.

78. The method of claim 77 wherein the immune response modifier molecule is selected from the group consisting of imidazoquinoline amines and 6,7-fused cycloalkylimidazopyridine amines, and pharmaceutically acceptable salts thereof.

79. The method of claim 75 wherein the disease is a neoplastic disease and the antigen is derived from neoplastic cells.

80. The method of claim 75 wherein the disease is caused by an infectious agent and the antigen is derived from the infectious agent.

81. The method of claim 75 wherein the antigen is recombinantly derived.

82. A method of preparing a cellular adjuvant for the treatment of a disease comprising:
(a) maturing plasmacytoid dendritic cells in vitro by treating the dendritic cells with an immune response modifier molecule that is an agonist of Toll-like receptor 6, Toll-like receptor 7 or Toll-like receptor 8; and
(b) exposing the mature dendritic cells to an antigen associated with said disease.

83. The method of claim 82 wherein the immune response modifier molecule is an agonist of Toll-like receptor 7.

84. The method of claim 82 wherein the immune response modifier molecule is selected from the group consisting of imidazoquinoline amines, imidazopyridine amines, 6,7-fused cycloalkylimidazopyridine amines, 1,2-bridged imidazoquinoline amines, thiazolo- and oxazolo-quinolinamines and pyridinamines, imidazonaphthyridine amines and tetrahydroimidazonaphthyridine amines, and pharmaceutically acceptable salts thereof.

85. The method of claim 84 wherein the immune response modifier molecule is selected from the group consisting of imidazoquinoline amines and 6,7-fused cycloalkylimidazopyridine amines, and pharmaceutically acceptable salts thereof.

86. The method of claim 82 wherein the disease is a neoplastic disease and the antigen is derived from neoplastic cells.

87. The method of claim 82 wherein the disease is caused by an infectious agent and the antigen is derived from the infectious agent.

88. The method of claim 82 wherein the antigen is recombinantly derived.

89. A method of treating a disease comprising administering a therapeutically effective dose of the cellular adjuvant of claim 82 to a mammal in need of such treatment.

90. A cellular adjuvant prepared by the method of claim 82.

91. A method of treating a disease comprising administering a therapeutically effective dose of plasmacytoid dendritic cells that have been matured by stimulation with an immune response modifier molecule that is an agonist of Toll-like receptor 6, Toll-like receptor 7 or Toll-like receptor 8 to mammal in need of such treatment.

92. The method of claim 91 wherein the disease is a neoplastic disease.

93. The method of claim 91 wherein the disease is a Th2-mediated disease.
